SEQUENCE LISTING (16 sequences)

SEQ ID NO: 1 - DNA, 3547 bp, homo sapiens
cccgggtcagcgcccgcccgcccgcgctcctcccggccgctcctcccgccccgcccggcccggcgccgactctgcggccgcccgacgagcccctcgcggcactgccccggccccggccccggccccggccccctcccgccgcaccgcccccggcccggccctccgccctccgcactcccgcctccctccctccgcccgctcccgcgccctcctccctccctcctccccagctgtcccgttcgcgtcatgccgagcctcccggccccgccggccccgctgctgctcctcgggctgctgctgctcggctcccggccggcccgcggcgccggcccagagccccccgtgctgcccatccgttctgagaaggagccgctgcccgttcggggagcggcaggctgcaccttcggcgggaaggtctatgccttggacgagacgtggcacccggacctaggggagccattcggggtgatgcgctgcgtgctgtgcgcctgcgaggcgcctcagtggggtcgccgtaccaggggccctggcagggtcagctgcaagaacatcaaaccagagtgcccaaccccggcctgtgggcagccgcgccagctgccgggacactgctgccagacctgcccccaggagcgcagcagttcggagcggcagccgagcggcctgtccttcgagtatccgcgggacccggagcatcgcagttatagcgaccgcggggagccaggcgctgaggagcgggcccgtggtgacggccacacggacttcgtggcgctgctgacagggccgaggtcgcaggcggtggcacgagcccgagtctcgctgctgcgctctagcctccgcttctctatctcctacaggcggctggaccgccctaccaggatccgcttctcagactccaatggcagtgtcctgtttgagcaccctgcagcccccacccaagatggcctggtctgtggggtgtggcgggcagtgcctcggttgtctctgcggctccttagggcagaacagctgcatgtggcacttgtgacactcactcacccttcaggggaggtctgggggcctctcatccggcaccgggccctggctgcagagaccttcagtgccatcctgactctagaaggccccccacagcagggcgtagggggcatcaccctgctcactctcagtgacacagaggactccttgcattttttgctgctcttccgagggctgctggaacccaggagtgggggactaacccaggttcccttgaggctccagattctacaccaggggcagctactgcgagaacttcaggccaatgtctcagcccaggaaccaggctttgctgaggtgctgcccaacctgacagtccaggagatggactggctggtgctgggggagctgcagatggccctggagtgggcaggcaggccagggctgcgcatcagtggacacattgctgccaggaagagctgcgacgtcctgcaaagtgtcctttgtggggctgatgccctgatcccagtccagacgggtgctgccggctcagccagcctcacgctgctaggaaatggctccctgatctatcaggtgcaagtggtagggacaagcagtgaggtggtggccatgacactggagaccaagcctcagcggagggatcagcgcactgtcctgtgccacatggctggactccagccaggaggacacacggccgtgggtatctgccctgggctgggtgcccgaggggctcatatgctgctgcagaatgagctcttcctgaacgtgggcaccaaggacttcccagacggagagcttcgggggcacgtggctgccctgccctactgtgggcatagcgcccgccatgacacgctgcccgtgcccctagcaggagccctggtgctaccccctgtgaagagccaagcagcagggcacgcctggctttccttggatacccactgtcacctgcactatgaagtgctgctggctgggcttggtggctcagaacaaggcactgtcactgcccacctccttgggcctcctggaacgccagggcctcggcggctgctgaagggattctatggctcagaggcccagggtgtggtgaaggacctggagccggaactgctgcggcacctggcaaaaggcatggcctccctgatgatcaccaccaagggtagccccagaggggagctccgagggcaggtgcacatagccaaccaatgtgaggttggcggactgcgcctggaggcggccggggccgagggggtgcgggcgctgggggctccggatacagcctctgctgcgccgcctgtggtgcctggtctcccggccctagcgcccgccaaacctggtggtcctgggcggccccgagaccccaacacatgcttcttcgaggggcagcagcgcccccacggggctcgctgggcgcccaactacgacccgctctgctcactctgcacctgccagagacgaacggtgatctgtgacccggtggtgtgcccaccgcccagctgcccacacccggtgcaggctcccgaccagtgctgccctgtttgccctgagaaacaagatgtcagagacttgccagggctgccaaggagccgggacccaggagagggctgctattttgatggtgaccggagctggcgggcagcgggtacgcggtggcaccccgttgtgcccccctttggcttaattaagtgtgctgtctgcacctgcaaggggggcactggagaggtgcactgtgagaaggtgcagtgtccccggctggcctgtgcccagcctgtgcgtgtcaaccccaccgactgctgcaaacagtgtccagtggggtcgggggcccacccccagctgggggaccccatgcaggctgatgggccccggggctgccgttttgctgggcagtggttcccagagagtcagagctggcacccctcagtgcccccttttggagagatgagctgtatcacctgcagatgtggggcaggggtgcctcactgtgagcgggatgactgttcactgccactgtcctgtggctcggggaaggagagtcgatgctgttcccgctgcacggcccaccggcggccagccccagagaccagaactgatccagagctggagaaagaagccgaaggctcttagggagcagccagagggccaagtgaccaagaggatggggcctgagctggggaaggggtggcatcgaggaccttcttgcattctcctgtgggaagcccagtgcctttgctcctctgtcctgcctctactcccacccccactacctctgggaaccacagctccacaagggggagaggcagctgggccagaccgaggtcacagccactccaagtcctgccctgccaccctcggcctctgtcctggaagccccacccctttcctcctgtacataatgtcactggcttgttgggatttttaatttatcttcactcagcaccaagggcccccgacactccactcctgctgcccctgagctgagcagagtcattattggagagttttgtatttattaaaacatttctttttcagtcaaaaaaaaaaaaaaaaaaaaaaaaaa

SEQ ID NO: 2 - DNA, 3561 bp, homo sapiens
cccgggtcagcgcccgcccgcccgcgctcctcccggccgctcctcccgccccgcccggcccggcgccgactctgcggccgcccgacgagcccctcgcggcactgccccggccccggccccggccccggccccctcccgccgcaccgcccccggcccggccctccgccctccgcactcccgcctccctccctccgcccgctcccgcgccctcctccctccctcctccccagctgtcccgttcgcgtcatgccgagcctcccggccccgccggccccgctgctgctcctcgggctgctgctgctcggctcccggccggcccgcggcgccggcccagagccccccgtgctgcccatccgttctgagaaggagccgctgcccgttcggggagcggcaggctgcaccttcggcgggaaggtctatgccttggacgagacgtggcacccggacctaggggagccattcggggtgatgcgctgcgtgctgtgcgcctgcgaggcgacagggaccttgaggcccagagagatgaagtagcttgtctagggtcacgcagcttcctcagtggggtcgccgtaccaggggccctggcagggtcagctgcaagaacatcaaaccagagtgcccaaccccggcctgtgggcagccgcgccagctgccgggacactgctgccagacctgcccccaggagcgcagcagttcggagcggcagccgagcggcctgtccttcgagtatccgcgggacccggagcatcgcagttatagcgaccgcggggagccaggcgctgaggagcgggcccgtggtgacggccacacggacttcgtggcgctgctgacagggccgaggtcgcaggcggtggcacgagcccgagtctcgctgctgcgctctagcctccgcttctctatctcctacaggcggctggaccgccctaccaggatccgcttctcagactccaatggcagtgtcctgtttgagcaccctgcagcccccacccaagatggcctggtctgtggggtgtggcgggcagtgcctcggttgtctctgcggctccttagggcagaacagctgcatgtggcacttgtgacactcactcacccttcaggggaggtctgggggcctctcatccggcaccgggccctggctgcagagaccttcagtgccatcctgactctagaaggccccccacagcagggcgtagggggcatcaccctgctcactctcagtgacacagaggactccttgcattttttgctgctcttccgagggctgctggaacccaggagtggggattctacaccaggggcagctactgcgagaacttcaggccaatgtctcagcccaggaaccaggctttgctgaggtgctgcccaacctgacagtccaggagatggactggctggtgctgggggagctgcagatggccctggagtgggcaggcaggccagggctgcgcatcagtggacacattgctgccaggaagagctgcgacgtcctgcaaagtgtcctttgtggggctgatgccctgatcccagtccagacgggtgctgccggctcagccagcctcacgctgctaggaaatggctccctgatctatcaggtgcaagtggtagggacaagcagtgaggtggtggccatgacactggagaccaagcctcagcggagggatcagcgcactgtcctgtgccacatggctggactccagccaggaggacacacggccgtgggtatctgccctgggctgggtgcccgaggggctcatatgctgctgcagaatgagctcttcctgaacgtgggcaccaaggacttcccagacggagagcttcgggggcacgtggctgccctgccctactgtgggcatagcgcccgccatgacacgctgcccgtgcccctagcaggagccctggtgctaccccctgtgaagagccaagcagcagggcacgcctggctttccttggatacccactgtcacctgcactatgaagtgctgctggctgggcttggtggctcagaacaaggcactgtcactgcccacctccttgggcctcctggaacgccagggcctcggcggctgctgaagggattctatggctcagaggcccagggtgtggtgaaggacctggagccggaactgctgcggcacctggcaaaaggcatggcctccctgatgatcaccaccaagggtagccccagaggggagctccgagggcaggtgcacatagccaaccaatgtgaggttggcggactgcgcctggaggcggccggggccgagggggtgcgggcgctgggggctccggatacagcctctgctgcgccgcctgtggtgcctggtctcccggccctagcgcccgccaaacctggtggtcctgggcggccccgagaccccaacacatgcttcttcgaggggcagcagcgcccccacggggctcgctgggcgcccaactacgacccgctctgctcactctgcacctgccagagacgaacggtgatctgtgacccggtggtgtgcccaccgcccagctgcccacacccggtgcaggctcccgaccagtgctgccctgtttgccctgagaaacaagatgtcagagacttgccagggctgccaaggagccgggacccaggagagggctgctattttgatggtgaccggagctggcgggcagcgggtacgcggtggcaccccgttgtgcccccctttggcttaattaagtgtgctgtctgcacctgcaaggggggcactggagaggtgcactgtgagaaggtgcagtgtccccggctggcctgtgcccagcctgtgcgtgtcaaccccaccgactgctgcaaacagtgtccagtggggtcgggggcccacccccagctgggggaccccatgcaggctgatgggccccggggctgccgttttgctgggcagtggttcccagagagtcagagctggcacccctcagtgcccccttttggagagatgagctgtatcacctgcagatgtggggcaggggtgcctcactgtgagcgggatgactgttcactgccactgtcctgtggctcggggaaggagagtcgatgctgttcccgctgcacggcccaccggcggccagccccagagaccagaactgatccagagctggagaaagaagccgaaggctcttagggagcagccagagggccaagtgaccaagaggatggggcctgagctggggaaggggtggcatcgaggaccttcttgcattctcctgtgggaagcccagtgcctttgctcctctgtcctgcctctactcccacccccactacctctgggaaccacagctccacaagggggagaggcagctgggccagaccgaggtcacagccactccaagtcctgccctgccaccctcggcctctgtcctggaagccccacccctttcctcctgtacataatgtcactggcttgttgggatttttaatttatcttcactcagcaccaagggcccccgacactccactcctgctgcccctgagctgagcagagtcattattggagagttttgtatttattaaaacatttctttttcagtcaaaaaaaaaaaaaa

SEQ ID NO: 3 - DNA, 3506 bp, homos sapiens
cccgggtcagcgcccgcccgcccgcgctcctcccggccgctcctcccgccccgcccggcccggcgccgactctgcggccgcccgacgagcccctcgcggcactgccccggccccggccccggccccggccccctcccgccgcaccgcccccggcccggccctccgccctccgcactcccgcctccctccctccgcccgctcccgcgccctcctccctccctcctccccagctgtcccgttcgcgtcatgccgagcctcccggccccgccggccccgctgctgctcctcgggctgctgctgctcggctcccggccggcccgcggcgccggcccagagccccccgtgctgcccatccgttctgagaaggagccgctgcccgttcggggagcggcaggctgcaccttcggcgggaaggtctatgccttggacgagacgtggcacccggacctaggggagccattcggggtgatgcgctgcgtgctgtgcgcctgcgaggcgcctcagtggggtcgccgtaccaggggccctggcagggtcagctgcaagaacatcaaaccagagtgcccaaccccggcctgtgggcagccgcgccagctgccgggacactgctgccagacctgcccccaggagcgcagcagttcggagcggcagccgagcggcctgtccttcgagtatccgcgggacccggagcatcgcagttatagcgaccgcggggagccaggcgctgaggagcgggcccgtggtgacggccacacggacttcgtggcgctgctgacagggccgaggtcgcaggcggtggcacgagcccgagtctcgctgctgcgctctagcctccgcttctctatctcctacaggcggctggaccgccctaccaggatccgcttctcagactccaatggcagtgtcctgtttgagcaccctgcagcccccacccaagatggcctggtctgtggggtgtggcgggcagtgcctcggttgtctctgcggctccttagggcagaacagctgcatgtggcacttgtgacactcactcacccttcaggggaggtctgggggcctctcatccggcaccgggccctggctgcagagaccttcagtgccatcctgactctagaaggccccccacagcagggcgtagggggcatcaccctgctcactctcagtgacacagaggactccttgcattttttgctgctcttccgagggctgctggaacccaggagtggggattctacaccaggggcagctactgcgagaacttcaggccaatgtctcagcccaggaaccaggctttgctgaggtgctgcccaacctgacagtccaggagatggactggctggtgctgggggagctgcagatggccctggagtgggcaggcaggccagggctgcgcatcagtggacacattgctgccaggaagagctgcgacgtcctgcaaagtgtcctttgtggggctgatgccctgatcccagtccagacgggtgctgccggctcagccagcctcacgctgctaggaaatggctccctgatctatcaggtgcaagtggtagggacaagcagtgaggtggtggccatgacactggagaccaagcctcagcggagggatcagcgcactgtcctgtgccacatggctggactccagccaggaggacacacggccgtgggtatctgccctgggctgggtgcccgaggggctcatatgctgctgcagaatgagctcttcctgaacgtgggcaccaaggacttcccagacggagagcttcgggggcacgtggctgccctgccctactgtgggcatagcgcccgccatgacacgctgcccgtgcccctagcaggagccctggtgctaccccctgtgaagagccaagcagcagggcacgcctggctttccttggatacccactgtcacctgcactatgaagtgctgctggctgggcttggtggctcagaacaaggcactgtcactgcccacctccttgggcctcctggaacgccagggcctcggcggctgctgaagggattctatggctcagaggcccagggtgtggtgaaggacctggagccggaactgctgcggcacctggcaaaaggcatggcctccctgatgatcaccaccaagggtagccccagaggggagctccgagggcaggtgcacatagccaaccaatgtgaggttggcggactgcgcctggaggcggccggggccgagggggtgcgggcgctgggggctccggatacagcctctgctgcgccgcctgtggtgcctggtctcccggccctagcgcccgccaaacctggtggtcctgggcggccccgagaccccaacacatgcttcttcgaggggcagcagcgcccccacggggctcgctgggcgcccaactacgacccgctctgctcactctgcacctgccagagacgaacggtgatctgtgacccggtggtgtgcccaccgcccagctgcccacacccggtgcaggctcccgaccagtgctgccctgtttgccctgagaaacaagatgtcagagacttgccagggctgccaaggagccgggacccaggagagggctgctattttgatggtgaccggagctggcgggcagcgggtacgcggtggcaccccgttgtgcccccctttggcttaattaagtgtgctgtctgcacctgcaaggggggcactggagaggtgcactgtgagaaggtgcagtgtccccggctggcctgtgcccagcctgtgcgtgtcaaccccaccgactgctgcaaacagtgtccagtggggtcgggggcccacccccagctgggggaccccatgcaggctgatgggccccggggctgccgttttgctgggcagtggttcccagagagtcagagctggcacccctcagtgcccccttttggagagatgagctgtatcacctgcagatgtggggcaggggtgcctcactgtgagcgggatgactgttcactgccactgtcctgtggctcggggaaggagagtcgatgctgttcccgctgcacggcccaccggcggccagccccagagaccagaactgatccagagctggagaaagaagccgaaggctcttagggagcagccagagggccaagtgaccaagaggatggggcctgagctggggaaggggtggcatcgaggaccttcttgcattctcctgtgggaagcccagtgcctttgctcctctgtcctgcctctactcccacccccactacctctgggaaccacagctccacaagggggagaggcagctgggccagaccgaggtcacagccactccaagtcctgccctgccaccctcggcctctgtcctggaagccccacccctttcctcctgtacataatgtcactggcttgttgggatttttaatttatcttcactcagcaccaagggcccccgacactccactcctgctgcccctgagctgagcagagtcattattggagagttttgtatttattaaaacatttctttttcagtcaaaaaaaaaaaaaa

SEQ ID NO: 4 - DNA, 2488 bp, homo sapiens
gacggagaaggccagtgcccaggttagtgagcagtgcccggcgcccgcttccctcacctccttttccagcctttgcacagcttgaaggttctgtcaccttttgcagtggtccaaatgagaaaaaagtggaaaatgggaggcatgaaatacatcttttcgttgttgttctttcttttgctagaaggaggcaaaacagagcaagtaaaacattcagagacatattgcatgtttcaagacaagaagtacagagtgggtgagagatggcatccttacctggaaccttatgggttggtttactgcgtgaactgcatctgctcagagaatgggaatgtgctttgcagccgagtcagatgtccaaatgttcattgcctttctcctgtgcatattcctcatctgtgctgccctcgctgcccagactccttacccccagtgaacaataaggtgaccagcaagtcttgcgagtacaatgggacaacttaccaacatggagagctgttcgtagctgaagggctctttcagaatcggcaacccaatcaatgcacccagtgcagctgttcggagggaaacgtgtattgtggtctcaagacttgccccaaattaacctgtgccttcccagtctctgttccagattcctgctgccgggtatgcagaggagatggagaactgtcatgggaacattctgatggtgatatcttccggcaacctgccaacagagaagcaagacattcttaccaccgctctcactatgatcctccaccaagccgacaggctggaggtctgtcccgctttcctggggccagaagtcaccggggagctcttatggattcccagcaagcatcaggaaccattgtgcaaattgtcatcaataacaaacacaagcatggacaagtgtgtgtttccaatggaaagacctattctcatggcgagtcctggcacccaaacctccgggcatttggcattgtggagtgtgtgctatgtacttgtaatgtcaccaagcaagagtgtaagaaaatccactgccccaatcgatacccctgcaagtatcctcaaaaaatagacggaaaatgctgcaaggtgtgtccaggtaaaaaagcaaaagaacttccaggccaaagctttgacaataaaggctacttctgcggggaagaaacgatgcctgtgtatgagtctgtattcatggaggatggggagacaaccagaaaaatagcactggagactgagagaccacctcaggtagaggtccacgtttggactattcgaaagggcattctccagcacttccatattgagaagatctccaagaggatgtttgaggagcttcctcacttcaagctggtgaccagaacaaccctgagccagtggaagatcttcaccgaaggagaagctcagatcagccagatgtgttcaagtcgtgtatgcagaacagagcttgaagatttagtcaaggttttgtacctggagagatctgaaaagggccactgttaggcaagacagacagtattggatagggtaaagcaagaaaactcaagctgcagctggactgcaggcttattttgcttaagtcaacagtgccctaaaactccaaactcaaatgcagtcaattattcacgccatgcacagcataatttgctcctttgtgtggagtggtgtgtcagcccttgaacatctcctccaaagagactagaagagtcttaaattatatgtgggaggaggagggatagaacatcacaacactgctctagtttcttggagaatcacatttctttacaggttaaagacaaacaagaccccagggtttttatctagaaagttattcaagtgaaagaaagagaagggaattgcttagtaggagttctgcagtatagaacaattacttgtatgaaattatacctttgaattttagaatgtcatgtgttcttttaaaaaaattagctccccatcctccctcctcactccctccctccctccttctctctctctctctctctccctccctctctcacagacacacacacacacacacacacacacacacacacgcacgtccacactcacattaaacgaaagctttatttgaagcaaagctagccaagattctacgttacttttcccttgactggatcccaagtagcttggaagtttttgtgcccaggagagtaaataactgtgaacaagaggctctgcccttaggtctttgtggctgtttaagtcaccaacaatagagtcagggtaaagaataaaaacactttcatagcctcattcattcacttagaagtggtaataatttttccctaatgataccacttttcttttccccctgtacctatgggacttccagaaagaagttaaattgagtaaaatcatcagaaactgaatccatgtaagaaaaaataattgttgaagaaagaagttgatagaattcaaaaaggccatctttttgctttcacatcaataaaatttaccaagtaataaaaaaaaaaaaaaaaaa

SEQ ID NO: 5 - DNA, 1311 bp, homo sapiens
cagcgaggagccggcgcctcccgcgccccgcggtcgccctggagtaatttcggatgcccagccgcggccgccttccccagtagacccgggagaggagttgcggccaacttgtgtgcctttcttccgccccggtgggagccggcgctgcgcgaagggctctcccggcggctcatgctgccggccctgcgcctgcccagcctcgggtgagccgcctccggagagacgggggagcgcggcggcgccgcgggctcggcgtgctctcctccggggacgcgggacgaagcagcagccccgggcgcgcgccagaggcatggagcgctgccccagcctaggggtcaccctctacgccctggtggtggtcctggggctgcgggcgacaccggccggcggccagcactatctccacatccgcccggcacccagcgacaacctgcccctggtggacctcatcgaacacccagaccctatctttgaccccaaggaaaaggatctgaacgagacgctgctgcgctcgctgctcgggggccactacgacccaggcttcatggccacctcgccccccgaggaccggcccggcgggggcgggggtgcagctgggggcgcggaggacctggcggagctggaccagctgctgcggcagcggccgtcgggggccatgccgagcgagatcaaagggctagagttctccgagggcttggcccagggcaagaagcagcgcctaagcaagaagctgcggaggaagttacagatgtggctgtggtcgcagacattctgccccgtgctgtacgcgtggaacgacctgggcagccgcttttggccgcgctacgtgaaggtgggcagctgcttcagtaagcgctcgtgctccgtgcccgagggcatggtgtgcaagccgtccaagtccgtgcacctcacggtgctgcggtggcgctgtcagcggcgcgggggccagcgctgcggctggattcccatccagtaccccatcatttccgagtgcaagtgctcgtgctagaactcgggggccccctgcccgcacccggacacttgatcgatccccaccgacgccccctgcaccgcctccaaccagttccaccaccctctagcgagggttttcaatgaacttttttttttttttttttttttttctgggctacagagacctagctttctggttcctgtaatgcactgtttaactgtgtaggaatgtatatgtgtgtgtatatacggtcccagttttaatttacttattaaaaggtcagtattatacgttaaaaaaaaaaaacccaaaaaaaaaaaaaaaaaaaaaaaaaaaaa

SEQ ID NO: 6 - protein, 14 aa, artificial
  artificial seqence of cystine knot
CXCXGXCXCXCXCX

SEQ ID NO: 7 - protein, 17 aa, artificial
  cystine knot motif
CXCXCXGXCXCXCXCXC

SEQ ID NO: 8 - protein, 19 aa, artificial
  cystine knot motif
CXCXCXCXCCXCXCXCCXC

SEQ ID NO: 9 - protein, 18 aa, artificial
  cystine knot motif
CXCXCXCXCXCXCXCCXC

SEQ ID NO: 10 - protein, 19 aa, artificial
  cystine knot motif
CXCXCXCXCXCXCXCXCXC

SEQ ID NO: 11 - DNA, 26 bp, artificial
  primer sequence
ctgcgggacttcgaggcgacacttct

SEQ ID NO: 12 - DNA, 26 bp, artificial
  primer sequence
tcttcctcctcctcctccccagactg

SEQ ID NO: 13 - DNA, 43 bp, artificial
  primer sequence
atggactgttattatgccttgttttctgtcaacaccatgattc

SEQ ID NO: 14 - DNA, 31 bp, artificial
  primer sequence
ccacgtatagtgaatggcgacggcagttctt

SEQ ID NO: 15 - DNA, 34 bp, artificial
  primer sequence
gtcaacaccatgattcctggtaaccgaatgctga

SEQ ID NO: 16 - DNA, 30 bp, artificial
  primer sequence
ttatacggtggaagccctgttcccagtcag

(19) United States
(12) Patent Application Publication
(10) Pub. No.: US 2006/0276385 A1
(43) Pub. Date: Dec. 7, 2006

(54) Anti-inflammatory agents and methods of their use

(76) Inventor: Hanjoong Jo, Marietta, GA (US)

Correspondence Address: THOMAS, KAYDEN, HORSTEMEYER & RISLEY, LLP, 100 GALLERIA PARKWAY, NW, STE 1750, ATLANTA, GA, 30339-5948, US

(21) Appl. No.: 10/541,953

(22) PCT Filed: Jan. 13, 2004
(86) PCT No.: PCT/US04/00759

§ 371 Date: Jul. 12, 2005

Related U.S. Application Data
(60) Provisional application No. 60/439,667, filed on Jan. 13, 2003.

(51) Int. Cl. A61K 48/00; A61K 38/17

(52) U.S. Cl. 514/12; 514/44

(57) ABSTRACT

The present disclosure provides compositions and methods for reducing or inhibiting vascular inflammation, for example inflammation resulting from unstable blood flow conditions such as oscillatory shear. Representative compositions include an antagonist of BMPs, for example BMP4, or BMP receptors, for example BMPR-I and/or BMPR-II, in an amount sufficient to for inhibiting or reducing vascular inflammation by interfering with binding of bone morphogenic protein or a fragment thereof to bone morphogenic protein receptors. Exemplary BMP antagonists include polypeptides having an eight-, nine-, or ten-membered ring cystine knot structure. Representative BMP antagonists include, but are not limited to the CAN family of proteins, the chordin family that includes chordin and ventroptin, and noggin.

### CROSS REFERENCE TO RELATED APPLICATION

This application claims benefit to U.S. Provisional Application No. 60/439,667 filed on Jan. 13, 2003, which is incorporated herein in its entirety.

### STATEMENT REGARDING FEDERALLY SPONSORED RESEARCH OR DEVELOPMENT

Aspects of the work disclosed herein were funded, in part, under Grant Nos. NAG 2-1431 awarded by NASA and 1RO1 HL67413-01 awarded by the National Institutes of Health. As a result, the government has certain rights in this invention.

### BACKGROUND

1. Technical Field

This disclosure is generally directed to compositions and methods for inhibiting or reducing bone morphogenic polypeptide signal transduction, more particularly, to compositions and methods for inhibiting inflammatory responses mediated by bone morphogenic polypeptides.

2. Related Art

Estimates by the American Heart Association for the year 2001 indicate that 64,400,000 Americans have one or more forms of cardiovascular disease (CVD). Coronary heart disease is caused by atherosclerosis, the narrowing of the coronary arteries due to fatty build ups of plaque. Endothelial cells of blood vessels are constantly exposed to shear stress (a dragging force generated by blood flow), which controls cellular structure and function such as regulation of vascular tone and diameter, vessel wall remodeling, hemostasis, and inflammatory responses (Davies, P. F., et al. (2002) Biorheology 39, 299-306). The importance of various types of shear stresses is highlighted by the focal development of atherosclerosis (Zarins, C. K., et al. (1983) Circ Res 53, 502-514). Atherosclerosis preferentially occurs in the arterial regions exposed to unstable shear stress conditions in branched or curved arteries, while straight arteries exposed to unidirectional laminar shear (LS) are relatively lesion-free (Ross, R. (1999) N Engl J Med 340, 115-126; Libby, P., et al. (2002) Circulation 105, 1135-1143; Garcia-Cardena, G., et al. (2001) Proc Natl Acad Sci USA 98,4478-4485). Atherosclerosis is now known as an inflammatory disease caused by endothelial dysfunction (Ross, R. (1999) N Engl J Med 340, 115-126; Libby, P., et al. (2002) Circulation 105, 1135-1143). One of the first visible markers of endothelial dysfunction in the lesion-prone areas is upregulation of inflammatory adhesion molecules such as E-selectin, vascular cell adhesion molecule-1 (VCAM-1) and ICAM-1 (Ross, R. (1999) N Engl J Med 340, 115-126; Libby, P., et al. (2002) Circulation 105, 1135-1143; Cybulsky, M. I., et al. (2001) J Clin Invest 107, 1255-1262; Endres, M., et al. (1997) Stroke 28, 77-82). These endothelial adhesion molecules play essential roles in adhesion and recruitment of monocytes to the subendothelial layer (Ross, R. (1999) N Engl J Med 340, 115-126; Libby, P.).

Current therapies for coronary heart disease include invasive procedures such as surgery as well as medications. Invasive procedures often have extensive recovery times and may not be indicated for all individuals. Medications, for example HMG CoA reductase inhibitors, are effective in some individuals, but not all. Moreover, currently available medications can have serious side effects. In addition, despite the recognition that atherosclerosis is an inflammatory disease, specific anti-inflammatory drugs in vasculature have not been developed.

Accordingly, there is a need for new compositions and methods for reducing, inhibiting, or blocking atherosclerosis and or vascular inflammation.

### BRIEF SUMMARY

It has been discovered that bone morphogenic proteins (BMPs), for example BMP4, act as inflammatory cytokines involved in inflammatory responses and atherogenesis. Aspects of the present disclosure are directed to compositions and methods for modulating BMP-induced inflammatory responses. One aspect provides compositions including an antagonist of BMPs, for example BMP4, or BMP receptors, for example BMPR-I and/or BMPR-II, in an amount sufficient for inhibiting or reducing vascular inflammation by interfering with binding of bone morphogenic protein or a fragment thereof to bone morphogenic protein receptors. The compositions optionally include an antioxidant, for example N-acetyl cysteine. Exemplary BMP antagonists include polypeptides having an eight-, nine-, or ten-membered ring cystine knot structure. Representative BMP antagonists include, but are not limited to the CAN family of proteins, the chordin family that includes chordin (SEQ. ID. NOs.: 1-3) and ventroptin (SEQ. ID. NO.: 4), and noggin (SEQ. ID. NO.: 5).

One aspect of the disclosed BMP antagonists includes a general eight-membered ring structure represented by the sequence Cys-Xₙ-Cys-X-Gly-X-Cys-X-Cys₁₋₂-Xₙ-Cys-X-Cys-X (SEQ. ID. NO.: 6) wherein in n represents any number. Another aspect provides antagonists having the sequence Cys-X₁₃-Cys-X₉₋₁₀-Cys-X-Gly-X-Cys-Xₙ-Cys-X₁₃-Cys-Xₙ-Cys-X-Cys (SEQ. ID. NO.: 7). Still another aspect provides antagonists having the sequence Cys-X₇-Cys-X₅-Cys-X₃-Cys-X₁₄-Cys-Cys-X-Cys-X₄-Cys-X9-Cys-Cys-X₂-Cys (SEQ. ID. NO.: 8). Yet another aspect provides antagonists having the sequence Cys-Xₙ-Cys-X₂-Cys-X-Cys-Xₙ-Cys-X₄₋₆-Cys-Xₙ-Cys-X₉-Cys-Cys-X₂-Cys (SEQ. ID. NO.: 9). Another aspect provides antagonists having the sequence Cys-X₂₂-Cys-X₅-Cys-X₇-Cys-X₁₄-Cys-X₇-Cys-X₁₄-Cys-X₇-Cys-X₁₂-Cys-X-Cys (SEQ. ID. NO.: 10).

The present disclosure also provides medical devices incorporating the disclosed antagonists. A representative device includes a vascular stent coated or impregnated with the disclosed antagonists. The device can be configured to be inserted into a blood vessel where it can release the disclosed antagonists to help reduce or prevent vascular inflammation, for example vascular inflammation resulting from oscillatory shear.

Another aspect of the disclosure provides polynucleotide compositions that encode the disclosed antagonists or modified BMPs. The modified BMPs can be engineered to bind to BMP receptors, for example vascular cell receptors, without triggering the BMP receptor signal transduction pathway. Another aspect includes inhibitory nucleic acid compositions specific for BMPs or BMP receptors.

Still other aspects of the disclosure provide methods for using the described antagonists. One aspect provides a method for reducing vascular inflammation by administering to a host an amount of bone morphogenic protein antagonist or bone morphogenic protein receptor antagonist sufficient or effective to inhibit binding of bone morphogenic protein or a fragment thereof to vascular cells of the host. Other methods include reducing the adhesion of monocytes to vascular cells by interfering with or inhibiting the BMP signal transduction pathway.

### BRIEF DESCRIPTION OF THE DRAWINGS

FIGS. 1A-C are photomicrographs of cells exposed to laminar shear, oscillating shear, or static conditions.

FIGS. 2A-C are bar graphs showing differential regulation of BMP4 mRNA and protein expression by LS or OS.

FIGS. 3A-F are photomicrographs of cells with antibodies specific for BMP4 or ICAM-1.

FIGS. 4A-C are bar graphs showing BMP4 stimulates monocyte adhesion to endothelial cells.

FIGS. 5A-E are bar graphs and blots showing BMP4 produced by OS selectively increases surface expression of ICAM-1, but not VCAM-1 and E-selectin.

FIG. 6 is a bar graph showing antibody specific for ICAM-1 inhibits OS induced monocyte binding.

FIGS. 7A and B are bar graphs showing BMP4 stimulates monocyte adhesion in an ICAM-1 and reactive oxygen species (ROS)-dependent manner in endothelial cells.

FIGS. 8A and B are bar graphs showing OS and BMP4 induce ICAM-1 expression in a ROS-dependent manner.

FIG. 9A is a Western blot showing the presence of p47phox in MAE-p47^{-/-} cells transfected with p47phox.

FIG. 9B is a bar graph showing BMP4 stimulates monocyte adhesion in a NADPH oxidase dependent manner.

FIGS. 10A and B are bar graphs showing BMP4 stimulates ROS (O₂⁻ and H₂O₂) production in MAEC.

FIG. 11 is a bar graph showing BMP4 antagonist, noggin, blocks OS-induced O₂⁻ production.

FIG. 12 is an illustration of BMP4 signal transduction pathway in vascular cells and the effects of shear conditions.

### DETAILED DESCRIPTION

### 1. DEFINITIONS

Unless otherwise indicated the following terms used in the specification and claims have the meanings discussed below:

The term “organism” or “host” refers to any living entity comprised of at least one cell. A living organism can be as simple as, for example, a single eukaryotic cell or as complex as a mammal, including a human being.

The term “therapeutically effective amount” as used herein refers to that amount of the compound being administered which will relieve to some extent one or more of the symptoms of the disorder being treated. In reference to vascular inflammation, a therapeutically effective amount refers to that amount which has the effect of (1) reducing the amount inflammation, (2) inhibiting (that is, slowing to some extent, preferably stopping) bone morphogenic protein signal transduction, (3) inhibiting (that is, slowing to some extent, preferably stopping) ICAM-1 expression, and/or, (4) relieving to some extent (or, preferably, eliminating) one or more symptoms associated with vascular inflammation or arteriosclerosis related disease.

“Pharmaceutically acceptable salt” refers to those salts which retain the biological effectiveness and properties of the free bases and which are obtained by reaction with inorganic or organic acids such as hydrochloric acid, hydrobromic acid, sulfuric acid, nitric acid, phosphoric acid, methanesulfonic acid, ethanesulfonic acid, p-toluenesulfonic acid, salicylic acid, malic acid, maleic acid, succinic acid, tartaric acid, citric acid, and the like.

A “pharmaceutical composition” refers to a mixture of one or more of the compounds described herein, or pharmaceutically acceptable salts thereof, with other chemical components, such as physiologically acceptable carriers and excipients. The purpose of a pharmaceutical composition is to facilitate administration of a compound to an organism.

As used herein, a “pharmaceutically acceptable carrier” refers to a carrier or diluent that does not cause significant irritation to an organism and does not abrogate the biological activity and properties of the administered compound.

An “excipient” refers to an inert substance added to a pharmaceutical composition to further facilitate administration of a compound. Examples, without limitation, of excipients include calcium carbonate, calcium phosphate, various sugars and types of starch, cellulose derivatives, gelatin, vegetable oils and polyethylene glycols.

“Treating” or “treatment” of a disease includes preventing the disease from occurring in an animal that may be predisposed to the disease but does not yet experience or exhibit symptoms of the disease (prophylactic treatment), inhibiting the disease (slowing or arresting its development), providing relief from the symptoms or side-effects of the disease (including palliative treatment), and relieving the disease (causing regression of the disease). With regard to cancer, these terms simply mean that the life expectancy of an individual affected with a cancer will be increased or that one or more of the symptoms of the disease will be reduced.

The term “prodrug” refers to an agent, including nucleic acids and proteins, which is converted into a biologically active form in vivo. Prodrugs are often useful because, in some situations, they may be easier to administer than the parent compound. They may, for instance, be bioavailable by oral administration whereas the parent compound is not. The prodrug may also have improved solubility in pharmaceutical compositions over the parent drug. A prodrug may be converted into the parent drug by various mechanisms, including enzymatic processes and metabolic hydrolysis. Harper, N. J. (1962). Drug Latentiation in Jucker, ed. Progress in Drug Research, 4:221-294; Morozowich et al. (1977). Application of Physical Organic Principles to Prodrug Design in E. B. Roche ed. Design of Biopharmaceutical Properties through Prodrugs and Analogs, APhA; Acad. Pharm. Sci.; E. B. Roche, ed. (1977). Bioreversible Carriers in Drug in Drug Design, Theory and Application, APhA; H. Bundgaard, ed. (1985) Design of Prodrugs, Elsevier; Wang et al. (1999) Prodrug approaches to the improved delivery of peptide drug, Curr. Pharm. Design. 5(4):265-287; Pauletti et al. (1997). Improvement in peptide bioavailability: Peptidomimetics and Prodrug Strategies, Adv. Drug. Delivery Rev. 27:235-256; Mizen et al. (1998). The Use of Esters as Prodrugs for Oral Delivery of β-Lactam antibiotics, Pharm. Biotech. 11,:345-365; Gaignault et al. (1996). Designing Prodrugs and Bioprecursors I. Carrier Prodrugs, Pract. Med. Chem. 671-696; M. Asgharnejad (2000). Improving Oral Drug Transport Via Prodrugs, in G. L. Amidon, P. I. Lee and E. M. Topp, Eds., Transport Processes in Pharmaceutical Systems, Marcell Dekker, p. 185-218; Balant et al. (1990) Prodrugs for the improvement of drug absorption via different routes of administration, Eur. J. Drug Metab. Pharmacokinet., 15(2): 143-53; Balimane and Sinko (1999). Involvement of multiple transporters in the oral absorption of nucleoside analogues, Adv. Drug Delivery Rev., 39(1-3):183-209; Browne (1997). Fosphenytoin (Cerebyx), Clin. Neuropharmacol. 20(1): 1-12; Bundgaard (1979). Bioreversible derivatization of drugs--principle and applicability to improve the therapeutic effects of drugs, Arch. Pharm. Chemi. 86(1): 1-39; H. Bundgaard, ed. (1985) Design of Prodrugs, New York: Elsevier; Fleisher et al. (1996). Improved oral drug delivery: solubility limitations overcome by the use of prodrugs, Adv. Drug Delivery Rev. 19(2): 115-130; Fleisher et al. (1985). Design of prodrugs for improved gastrointestinal absorption by intestinal enzyme targeting, Methods Enzymol. 112: 360-81; Farquhar D, et al. (1983). Biologically Reversible Phosphate-Protective Groups, J. Pharm. Sci., 72(3): 324-325; Han, H. K. et al. (2000). Targeted prodrug design to optimize drug delivery, AAPS Pharm Sci., 2(1): E6; Sadzuka Y. (2000). Effective prodrug liposome and conversion to active metabolite, Cur.r Drug Metab., 1(1):31-48; D. M. Lambert (2000) Rationale and applications of lipids as prodrug carriers, Eur. J. Pharm. Sci., 11 Suppl 2:S15-27; Wang, W. et al. (1999) Prodrug approaches to the improved delivery of peptide drugs. Curr. Pharm. Des., 5(4):265-87.

Bone morphogenic protein signal transduction pathway means the physiological and or biological sequence of events and the proteins and genes involved therein, from the production of bone morphogenic protein or bone morphogenic protein receptors through the binding to the receptor and the subsequent binding and activation of the receptor and associated proteins and cofactors resulting in the induction of gene transcription.

The term “nucleic acid” is a term of art that refers to a string of at least two base-sugar-phosphate combinations. For naked DNA delivery, a polynucleotide contains more than 120 monomeric units since it must be distinguished from an oligonucleotide. However, for purposes of delivering RNA, RNAi and siRNA, either single or double stranded, a polynucleotide contains 2 or more monomeric units. Nucleotides are the monomeric units of nucleic acid polymers. The term includes deoxyribonucleic acid (DNA) and ribonucleic acid (RNA) in the form of a messenger RNA, anti-sense, plasmid DNA, parts of a plasmid DNA or genetic material derived from a virus. Anti-sense is a polynucleotide that interferes with the function of DNA and/or RNA. The term nucleic acids—refers to a string of at least two base-sugar-phosphate combinations. Natural nucleic acids have a phosphate backbone, artificial nucleic acids may contain other types of backbones, but contain the same bases. Nucleotides are the monomeric units of nucleic acid polymers. The term includes deoxyribonucleic acid (DNA) and ribonucleic acid (RNA). RNA may be in the form of an tRNA (transfer RNA), snRNA (small nuclear RNA), rRNA (ribosomal RNA), mRNA (messenger RNA), anti-sense RNA, RNAi, siRNA, and ribozymes. The term also includes PNAs (peptide nucleic acids), phosphorothioates, and other variants of the phosphate backbone of native nucleic acids.

The term “siRNA” means a small inhibitory ribonucleic acid. The siRNA are typically less than 30 nucleotides in length and can be single or double stranded. The ribonucleotides can be natural or artificial and can be chemically modified. Longer siRNAs can comprise cleavage sites that can be enzymatically or chemically cleaved to produce siRNAs having lengths less than 30 nucleotides, typically 21 to 23 nucleotides. siRNAs share sequence homology with corresponding target mRNAs. The sequence homology can be 100 percent or less but sufficient to result is sequence specific association between the siRNA and the targeted mRNA. Exemplary siRNAs do not activate the interferon signal transduction pathway.

The term “inhibitory nucleic acid” means an RNA, DNA, or combination thereof that interferes or interrupts the translation of mRNA. Inhibitory nucleic acids can be single or double stranded. The nucleotides of the inhibitory nucleic acid can be chemically modified, natural or artificial.

The term “polypeptide” or “protein”, as used herein, includes all polypeptides as described below. The basic structure of polypeptides is well known and has been described in innumerable textbooks and other publications in the art. In this context, the term is used herein to refer to any peptide or protein comprising two or more amino acids joined to each other in a linear chain by peptide bonds. As used herein, the term refers to both short chains, which also commonly are referred to in the art as peptides, oligopeptides and oligomers, for example, and to longer chains, which generally are referred to in the art as proteins, of which there are many types.

It will be appreciated that polypeptides often contain amino acids other than the 20 amino acids commonly referred to as the 20 naturally occurring amino acids, and that many amino acids, including the terminal amino acids, may be modified in a given polypeptide, either by natural processes, such as processing and other post-translational modifications, but also by chemical modification techniques which are well known to the art. Typical modifications that occur naturally in polypeptides are numerous, and they are well described in the art, and they are well known to those of skill in the art.

Among the known modifications which may be present in polypeptides of the present are, to name an illustrative few, acetylation, acylation, ADP-ribosylation, amidation, covalent attachment of flavin, covalent attachment of a heme moiety, covalent attachment of a nucleotide or nucleotide derivative, covalent attachment of a lipid or lipid derivative, covalent attachment of phosphotidylinositol, cross-linking, cyclization, disulfide bond formation, demethylation, formation of covalent cross-links, formation of cystine, formation of pyroglutamate, formylation, gamma-carboxylation, glycosylation, GPI anchor formation, hydroxylation, iodination, methylation, myristoylation, oxidation, proteolytic processing, phosphorylation, prenylation, racemization, selenoylation, sulfation, transfer-RNA mediated addition of amino acids to proteins such as arginylation, and ubiquitination.

Such modifications are well known to those of skill and have been described in great detail in the scientific literature. Several particularly common modifications, glycosylation, lipid attachment, sulfation, γ-carboxylation of glutamic acid residues, hydroxylation and ADP-ribosylation, for instance, are described in most basic texts, such as, for instance Proteins-Structure and Molecular Properties, 2nd Ed., T. E. Creighton, W. H. Freeman and Company, New York (1993). Many detailed reviews are available on this subject, such as, for example, those provided by Wold, F., Posttranslational Protein Modifications: Perspectives and Prospects, pgs. 1-12 In Posttranslational Covalent Modification of Proteins, B. C. Johnson, Ed., Academic Press, New York (1983); Seifter et al., Analysis For Protein Modifications and Nonprotein Cofactors, Meth. Enzymol. 182: 626-646 (1990) and Rattan et al., Protein Synthesis: Posttranslational Modifications and Aging, Ann. N.Y. Acad. Sci. 663: 48-62 (1992).

It will be appreciated, as is well known and as noted above, that polypeptides are not always entirely linear. For instance, polypeptides may be branched as a result of ubiquitination, and they may be circular, with or without branching, generally as a result of posttranslational events, including natural processing event and events brought about by human manipulation which do not occur naturally. Circular, branched and branched circular polypeptides may be synthesized by non-translation natural process and by entirely synthetic methods, as well.

Modifications can occur anywhere in a polypeptide, including the peptide backbone, the amino acid side-chains and the amino or carboxyl termini. In fact, blockage of the amino or carboxyl group in a polypeptide, or both, by a covalent modification, is common in naturally occurring and synthetic polypeptides and such modifications may be present in polypeptides of the present invention, as well. For instance, the amino terminal residue of polypeptides made in E. coli, prior to proteolytic processing, almost invariably will be N-formylmethionine.

The modifications that occur in a polypeptide often will be a function of how it is made. For polypeptides made by expressing a cloned gene in a host, for instance, the nature and extent of the modifications in large part will be determined by the host cell posttranslational modification capacity and the modification signals present in the polypeptide amino acid sequence. For instance, as is well known, glycosylation often does not occur in bacterial hosts such as E. coli. Accordingly, when glycosylation is desired, a polypeptide should be expressed in a glycosylating host, generally a eukaryotic cell. Insect cells often carry out the same posttranslational glycosylations as mammalian cells and, for this reason, insect cell expression systems have been developed to express efficiently mammalian proteins having native patterns of glycosylation, inter alia. Similar considerations apply to other modifications.

It will be appreciated that the same type of modification may be present in the same or varying degree at several sites in a given polypeptide. Also, a given polypeptide may contain many types of modifications.

In general, as used herein, the term polypeptide encompasses all such modifications, including those that are present in polypeptides synthesized by expressing a polynucleotide in a host cell.

A variant of polynucleotides or polypeptides, as the term is used herein, are polynucleotides or polypeptides that differ from a reference polynucleotide or polypeptide, respectively. A variant and reference polypeptide may differ in amino acid sequence by one or more substitutions, additions, deletions, fusions and truncations, which may be present in any combination. Variants include substitution of amino acids wherein the substituted amino acid has no effect on the biological activity of a BMP protein antagonist or BMP protein receptor antagonist.

### 2. EMBODIMENTS

It has been discovered that bone morphogenic proteins, for example BMP4, act as mechanosensitive and pro-inflammatory agents in, for example, vascular tissue. A first embodiment of the present disclosure provides compositions, for example pharmaceutical compositions, including a bone morphogenic protein antagonist, a bone morphogenic protein receptor antagonist, or a prodrug thereof in an amount sufficient for inhibiting vascular inflammation, for example, by interfering with binding of bone morphogenic protein or a fragment thereof to bone morphogenic protein receptors. The compositions optionally include an antioxidant, for example N-acetyl cysteine. The term “antagonist” means a substance that interferes with, reduces, and/or blocks the biological action of another substance. Antagonist includes naturally occurring substances as well as artificial or manufactured substances. One embodiment provides an BMP antagonist including an antibody specific for BMP4, BMP receptors, or a fragment thereof. The antibodies can be poly- or monoclonal, humanized, or chimeric.

Other BMP antagonists of the present disclosure include polypeptides having a cysteine arrangement consistent with the formation of the cystine knot structure. Exemplary BMP antagonists having a cystine knot structure include, but are not limited to, the CAN family of proteins having an eight-membered ring cystine knot structure, the twisted gastrulation protein having a nine-membered ring cystine knot structure, the chordin family that includes chordin and ventroptin having a ten-membered ring type I cystine knot structure, and noggin having a ten-membered ring type II cystine knot structure.

BMP antagonists of the present disclosure can include a general eight-membered ring structure represented by Cys-Xₙ-Cys-X-Gly-X-Cys-X-Cys₁₋₂-Xₙ-Cys-X-Cys-X (SEQ. ID. NO.: 6) wherein in n represents any number. One embodiment provides antagonists having Cys-X₁₃-Cys-X₉₋₁₀-Cys-X-Gly-X-Cys-Xₙ-Cys-X₁₃-Cys-Xₙ-Cys-X-Cys (SEQ. ID. NO.: 7). Another embodiment provides antagonists having Cys-X₇-Cys-X₅-Cys-X₃-Cys-X₁₄-Cys-Cys-X-Cys-X₄-Cys-X₉-Cys-Cys-X₂-Cys (SEQ. ID. NO.: 8). Still another embodiment provides antagonists having Cys-Xₙ-Cys-X₂-Cys-X-Cys-Xₙ-Cys-X₄₋₆-Cys-Xₙ-Cys-X₉-Cys-Cys-X₂-Cys (SEQ. ID. NO.: 9). Yet another embodiment provides antagonists having Cys-X₂₂-Cys-X₅-Cys-X₇-Cys-X₁₄-Cys-X₇-Cys-X₁₄-Cys-X₇-Cys-X₁₂-Cys-X-Cys (SEQ. ID. NO.: 10).

Noggin is a small glycoprotein (32 kD) produced as a homodimer, whereas chordin is a large protein (120 kD). Noggin contains a carboxy-terminal cysteine-rich domain. Chordin contains cysteine-rich repeats similar to those found in TSP-1, procollagens I and III, and von Willebrand factor. Although not structurally related, both chordin and noggin bind specifically to BMPs, but not to activin or TGF, and antagonize BMP signaling by blocking BMP interaction with cell-surface receptors (Piccolo et al. (1996) Cell 86:589-98; Zimmerman et al. (1996) Cell 86:599-606).

Noggin inhibits BMPs by blocking the surfaces required to interact with type I and II BMP receptors. The N-terminal segment of each noggin monomer adopts an extended conformation and wraps around a BMP monomer directly occupying the receptor contact sites (Groope et al. (2002) Nature 420, 636-642) (Shi, Yigong and Joan Massague (2003) Cell 113, 685-700).

Exemplary BMP antagonists disclosed herein include fragments of the above-referenced proteins, in particular the N-terminal fragment or the carboxy-terminal. One embodiment includes an antagonist including the N-terminal or carboxy terminal fragment of noggin. Such fragments are of sufficient length to preserve binding of the fragment to a BMP, but prevent or inhibit the activation of the BMP signal transduction pathway through a BMP receptor via a BMP-antagonist complex. Suitable fragments, for example N-terminal fragments, are about 10 to about 200 residues, more typically about 50 to about 150 residues, even more typically about 100 residues in length.

2.1 Bone Morphogenic Proteins and Receptors

BMPs are members of the transforming growth factory-β (TGF-β) superfamily and are involved in bone formation, embryonic development and differentiation (Massague, J. (2000) Nat Rev Mol Cell Biol 1, 169-178; Hogan, B. L. (1996) Curr Opin Genetics Dev 6, 432-438). There are two types of signaling receptors specific for BMPs: BMPR-I and BMPR-II, and it appears that they are both required for signaling (Dale, L., and Jones, C. M. (1999) Bioessays 21, 751-760). Antagonists of the present disclosure can be specific for BMPR-I or BMPR-II, or can antagonize both receptors types. Three BMP type I receptors, BMPR-IA (also known as ALK3, Activin-Like Kinase-3), BMPR-IB (ALK6), and ALK2 and one BMP type II receptor have been identified (Kawabata, M., et al. (1998) Cytokine Growth Factor Rev 9, 49-61). Although somewhat variable depending upon species and vascular bed-origins, endothelial cells from mouse arteries as well as cultured murine and bovine aortic endothelial cells have been shown to express both type I (ALK2, 3, and 6) and type II BMPR's (Valdimaisdottir, G., et al. (2002) Circulation 106, 2263-2270). Unlike their well-known effects in bone formation and embryonic development, the functional importance of BMPR's in vascular wall is not clear. One notable exception is the link in vascular smooth muscle cells as demonstrated by the loss-of-function mutations of the type II BMPR in familial primary pulmonary hypertension and sporadic primary pulmonary hypertension (De Caestecker, M., and Meyrick, B, (2001) Respir Res 2, 193-197). In endothelial cells, transfection with constitutively active mutants of ALK2, ALK3, and ALK6 has been shown to stimulate expression of K/gene and angiogenic responses (Valdimaisdottir, G., et al. (2002) Circulation 106, 2263-2270). BMP4 has now been shown to induce inflammatory responses, especially in endothelial cells.

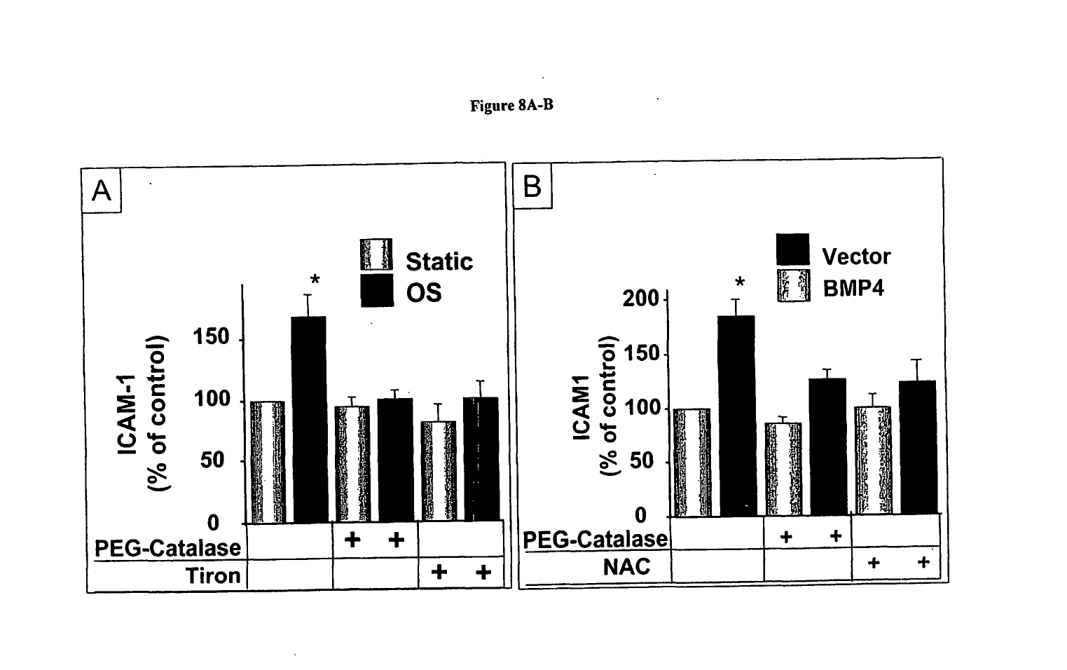

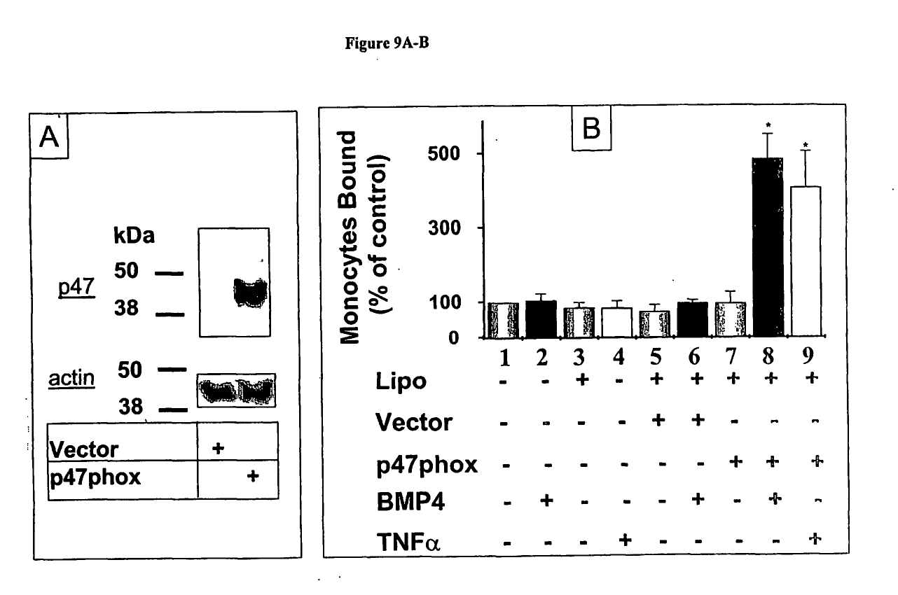

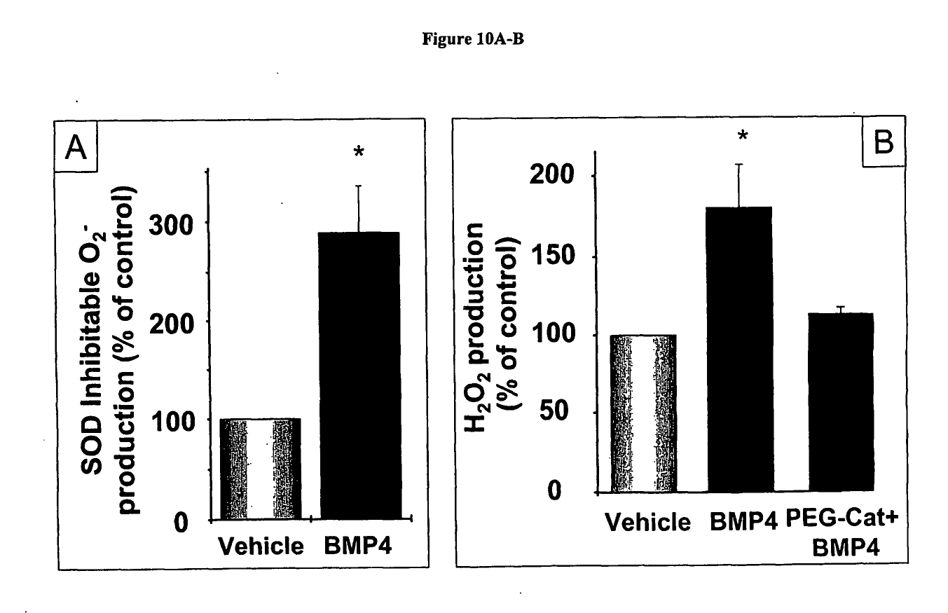

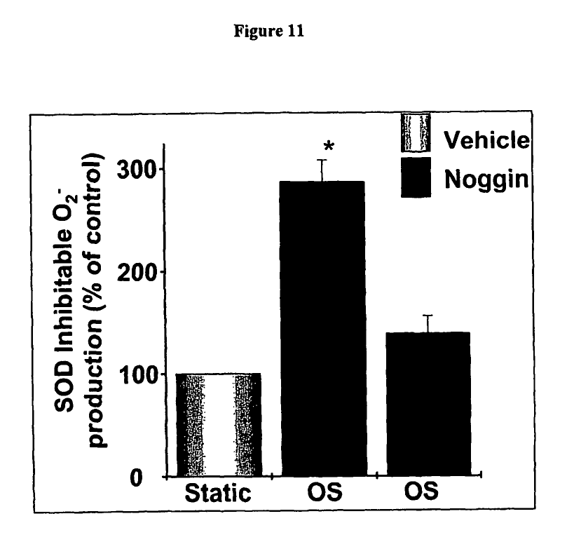

It has also been discovered that OS stimulates NAD(P)H oxidases to produce radical oxygen species (ROS) such as O₂⁻ and its metabolite H₂O₂. In addition, ROS was discovered to be responsible for subsequent monocyte adhesion response. BMP4-dependent monocyte adhesion was restored to the MAE-p47^{-/-} cells by over expressing p47phox (FIG. 9B). More over, OS induced O₂⁻ production was blocked by using the BMP antagonist, noggin (FIG. 11). BMP4 alone can stimulate O₂⁻ and H₂O₂ production in endothelial cells (FIG. 10A and 10B). The data disclosed herein indicated that BMP4 is the gene product induced in response to OS and that it is responsible for triggering the ROS production and the subsequent monocyte adhesion responses, for example in vascular cells.

It has also been discovered that BMP4 stimulates monocyte adhesion by increasing surface expression of ICAM-1 in endothelial cells. Accordingly, embodiments of the present disclosure are directed to methods and compositions that inhibit or block BMP4 biological activity, for example inhibit BMP4 signal transduction via BMPR-I or BMPR-II receptor types. By interfering with the signal transduction of BMP4, for example in vascular cells, ROS production can be reduced and ICAM-1 expression can be inhibited or reduced thereby inhibiting or reducing a vascular inflammation response such as monocyte adhesion.

It has been further discovered that OS and BMP4 selectively regulate expression of ICAM-1 without significantly affecting VCAM-1 and E-selectin. While both VCAM-1 and ICAM-1 are important in the pathogenesis of atherosclerosis, ICAM-1 expression in the lesion-prone areas seems to be regulated mainly by oscillatory shear stress, while VCAM-1 seems to be more responsive to high cholesterolemic conditions. Thus, embodiments of the present disclosure are directed to compositions and methods for treating vascular inflammation due, at least in part, to oscillatory shear stress.

VCAM-1 expression and monocyte adhesion are kept very low in most healthy arteries for unknown mechanisms (Endres, M., et al. (1997) Stroke 28, 77-82; Nakashima, Y., et al. (1998) Arterioscler Thromb Vase Biol 18, 842-851). Chronic exposure of endothelial cells to LS (a physiological condition expected for healthy straight arteries in vivo) virtually eliminated VCAM-1 expression. This may be an important mechanism by which LS acts as a potent anti-inflammatory and anti-atherogenic force. The data presented herein indicates that LS inhibits monocyte adhesion by multiple mechanisms including the direct inhibition of VCAM-1 expression as well as downregulation of BMP4 protein, dis-inhibiting its effect on ICAM-1 expression. The inhibitory effect of LS on VCAM-1 has been shown to be regulated by the NO-dependent mechanisms (Tsao, P. S., et al. (1995) Circulation 92, 3513-3519).

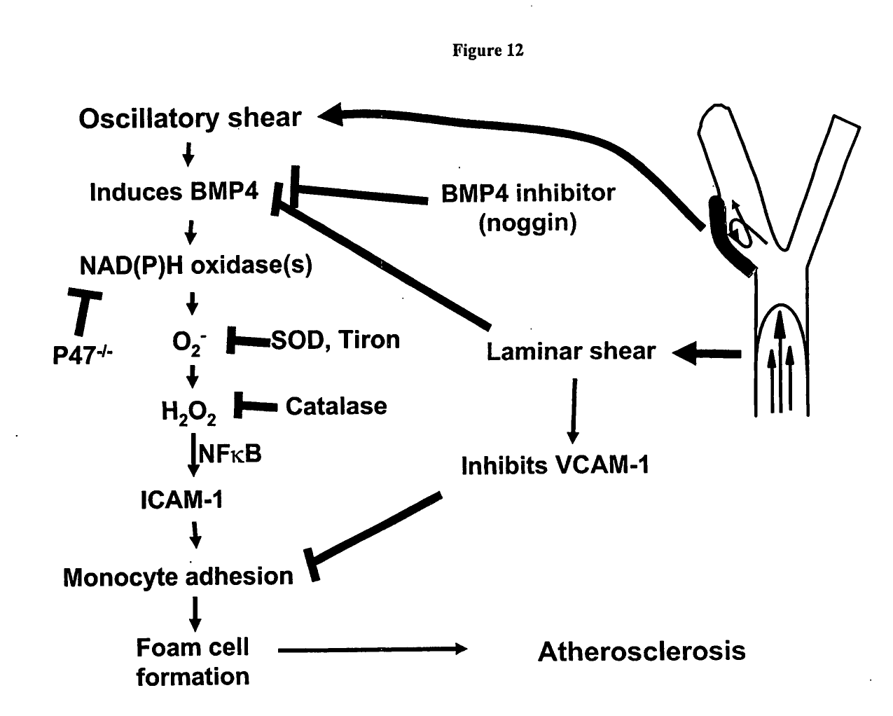

BMP4 plays a role as a mechanosensitive and pro-inflammatory cytokine mediating the opposite effects of OS and LS as described in FIG. 12. The endothelial cells in lesion-prone areas (indicated as a dark line in FIG. 12) experience low and disturbed shear stress including OS, which induces endothelial BMP4 expression. BMP4 stimulates NAD(P)H oxidases to produce ROS. ROS then initiates inflammatory cascades in an NFκB-dependent manner. For example, ICAM-1 surface expression in those activated endothelial cells is increased. ICAM-1 expression allows monocyte adhesion, foam cell lesion formation, and eventually leading to atherosclerotic plaque development. In contrast, LS acts as a potent anti-inflammatory and anti-atherogenic force by not only inhibiting BMP4 expression but also directly downregulating VCAM-l expression. Interestingly, it has been shown previously that LS induces expression of two signaling molecules, SMAD 6 and 1, which are known to inhibit BMP4 action (Topper, J. N., et al. (1997) Proc Natl Acad Sci USA 94, 9314-9319), providing an additional mechanism by which LS prevents BMP4-dependent responses.

2.2 Medical Devices

Other embodiments disclose medical devices that include bone morphogenic antagonists, bone morphogenic protein receptor antagonists, or a combination thereof. The disclosed antagonists can be coated on the surface of the medical device or the device can be saturated with the antagonists such that the antagonists are released from the device, for example over a period of time. Exemplary medical devices including the antagonists disclosed herein include, but are not limited to, vascular medical devices such as vascular stents.

Stents are typically used to prevent restenosis or treat an aneurysm without requiring surgery. Suitable stents include short flexible cylinders or scaffolds, made of metal or polymers, are often placed into a vessel to maintain or improve blood flow. Various types of these devices are widely used for reinforcing diseased blood vessels, for opening occluded blood vessels, and for defining an internal lumen to relieve pressure in an aneurysm. The stents allow blood to flow through the vessels at an improved rate while providing the desired lumen opening or structural integrity lost by the damaged vessels. Some stents are expanded to the proper size by inflating a balloon catheter, referred to as “balloon expandable” stents, while others are designed to elastically resist compression in a “self-expanding” manner.

Balloon expandable stents and self-expanding stents are generally delivered in a cylindrical form, crimped to a smaller diameter and are placed within a vessel using a catheter-based delivery system. When positioned at a desired site within a vessel, these devices are expanded by a balloon, or allowed to “self-expand,” to the desired diameter.

The antagonists to be delivered may be inserted into reservoirs of the device, as a liquid solution or gel, or they may be encapsulated within or by a release system. As used herein, “release system” includes both the situation where the antagonists are in pure form, as either a solid or liquid, or are in a matrix formed of degradable material or a material which releases incorporated molecules by diffusion out of or disintegration of the matrix. The antagonists can be sometimes contained in a release system because the degradation, dissolution, or diffusion properties of the release system provide a method for controlling the release rate of the molecules. The antagonists can be homogeneously or heterogeneously distributed within the release system. Selection of the release system is dependent on the desired rate of release of the molecules. Both non-degradable and degradable release systems can be used for delivery of molecules. Suitable release systems include polymers and polymeric matrices, non-polymeric matrices, or inorganic and organic excipients and diluents such as, but not limited to, calcium carbonate and sugar. Release systems may be natural or synthetic, although synthetic release systems typically are preferred due to the better characterization of release profiles.

The release system is selected based on the period over which release is desired. In the case of applications outside of the body, the release times may range from a fraction of a second to several months. In contrast, release times for in vivo applications, such as stent drug delivery, generally are within the range of several minutes to a year. In some cases, continuous (constant) release from a reservoir may be most useful. In other cases, a pulse (bulk) release from a reservoir may provide more effective results. A single pulse from one reservoir can be transformed into pulsatile release by using multiple reservoirs. It is also possible to incorporate several layers of a release system and other materials into a single reservoir to achieve pulsatile delivery from a single reservoir. Continuous release can be achieved by incorporating a release system that degrades, dissolves, or allows diffusion of molecules through it over an extended period of time. In addition, continuous release can be simulated by releasing several pulses of molecules in quick succession.

The release system material can be selected so that antagonists of various molecular weights are released from a reservoir by diffusion out of or through the material or by degradation of the material. In one embodiment for the technology outside of the body, the degradation or disintegration of the release system may occur by increasing its equilibrium vapor pressure causing the release system to evaporate, thereby releasing the molecules. This can be achieved by actively increasing the temperature of the release system with thin film resistors or passively through chemical interactions with the carrier liquids and/or gases. In the case of in vivo applications, it may be preferred that biodegradable polymers, bioerodible hydrogels, and protein delivery systems are used for the release of molecules by diffusion, degradation, or dissolution. In general, these materials degrade or dissolve either by enzymatic hydrolysis or exposure to water, or by surface or bulk erosion. Representative synthetic, biodegradable polymers include: poly(amides) such as poly(amino acids) and poly(peptides); poly(esters) such as poly(lactic acid), poly(glycolic acid), poly(lactic-co-glycolic acid), and poly(caprolactone); poly(anhydrides); poly(orthoesters); poly(carbonates); and chemical derivatives thereof (substitutions, additions of chemical groups, for example, alkyl, alkylene, hydroxylations, oxidations, and other modifications routinely made by those skilled in the art), copolymers and mixtures thereof. Representative synthetic, non-degradable polymers include: poly(ethers) such as poly(ethylene oxide), poly(ethylene glycol), and poly(tetramethylene oxide); vinyl polymers--poly(acrylates) and poly(methacrylates) such as methyl, ethyl, other alkyl, hydroxyethyl methacrylate, acrylic and methacrylic acids, and others such as poly(vinyl alcohol), poly(vinyl pyrolidone), and poly(vinyl acetate); poly(urethanes); cellulose and its derivatives such as alkyl, hydroxyalkyl, ethers, esters, nitrocellulose, and various cellulose acetates; poly(siloxanes); and any chemical derivatives thereof (substitutions, additions of chemical groups, for example, alkyl, alkylene, hydroxylations, oxidations, and other modifications routinely made by those skilled in the art), copolymers and mixtures thereof.

The device can be made of a durable material, for example biocompatible polymers, metals, mesh, alloys, composites, laminates, and the like. Alternatively, the device can be made of a material capable of being absorbed by an organism. Additionally, some embodiments provide implantable devices, such as stents that can be placed within a blood vessel.

2.3 Inhibitory Nucleic Acids

The inhibitory nucleic acids of certain embodiments of the present disclosure are directed to inhibiting or interfering with the expression of proteins involved in the bone morphogenic protein signal transduction pathway, in particular the bone morphogenic protein signal transduction pathway in vascular cells. The inhibitory nucleic acids disclosed herein include antisense nucleic acids and small inhibitory ribonucleic acids (siRNAs) specific for BMP, for example BMP4, or BMP receptors. siRNAs are typically less than 30 nucleotides in length, more typically 21 to 23 nucleotides in length, and can be single or double stranded. One strand of a double-stranded siRNA comprises at least a partial sequence complementary to a target mRNA. The ribonucleotides of the siRNA can be natural or artificial and can be chemically modified. Longer siRNAs can comprise cleavage sites that can be enzymatically or chemically cleaved to produce siRNAs having lengths less than 30 nucleotides. siRNAs share sequence homology with corresponding target mRNAs. The phosphate backbones of the siRNAs can be chemically modified to resist enzymatic degradation. The sequence homology can be about 100 percent or less, but sufficient to result is sequence specific association between the siRNA and the targeted mRNA.

Nucleic acids, in particular RNA, are known to participate in a form of post-transcriptional gene silencing termed “RNA interference” or RNAi. First observed in plants, reduction of expression of specific mRNA sequences was found to be inducible in Drosophila melanogaster and Caenorhabditis elegans by introduction of double-stranded RNA (dsRNA) molecules mimicking the sequence of the mRNA. The effect was found to be potent and extremely long-lived in these experimental model organisms, generally extending to the F1 progeny of a treated adult specimen. Additionally, the effect was found to be exquisitely sequence-specific; discrepancy of even a few base pairs between the dsRNA and the target mRNA virtually abolished the silencing. RNAi has been used experimentally in these non-mammalian systems to generate transient silencing of specific genes of interest, especially those which are not amenable to more traditional gene knockout methods (e.g., those that produce embryonic lethality and thus cannot be studied in the adult animal).

The first evidence that dsRNA could lead to gene silencing came from work in the nematode Caenorhabditis elegans. Researchers Guo and Kemphues used antisense RNA to shut down expression of the par-1 gene in order to assess its function. As expected, injection of the antisense RNA disrupted expression of par-1, but quizzically, injection of the sense-strand control did too. This result was a puzzle until three years later. It was then that Fire and Mello first injected dsRNA—a mixture of both sense and antisense strands—into C. elegans. This injection resulted in much more efficient silencing than injection of either the sense or the antisense strands alone. Injection of just a few molecules of dsRNA per cell was sufficient to completely silence the homologous gene's expression. Furthermore, injection of dsRNA into the gut of the worm caused gene silencing not only throughout the worm, but also in its first generation offspring. The potency of RNAi inspired Fire and Timmons to try feeding nematodes bacteria that had been engineered to express dsRNA homologous to the C. elegans unc-22 gene. Surprisingly, these worms developed an unc-22 null-like phenotype. Further work showed that soaking worms in dsRNA was also able to induce silencing. These strategies, whereby large numbers of nematodes are exposed to dsRNA, have enabled large-scale screens to select for RNAi-defective C. elegans mutants and have led to large numbers of gene knockout studies within this organism. Thus, one embodiment of the present disclosure provides siRNAs comprising a sense strand and an anti-sense strand, wherein the sense strand comprises at least a partial sequence of a target mRNA

RNAi has also been observed in Drosophila melanogaster. Although a strategy in which yeast were engineered to produce dsRNA and then fed to fruit flies failed to work, microinjecting Drosophila embryos with dsRNA does induce silencing. Silencing can also be induced by biolistic techniques in which dsRNA is “shot” into Drosophila embryos, or by engineering flies to carry DNA containing an inverted repeat of the gene to be silenced. Over the last few years, these RNAi strategies have been used as reverse genetics tools in Drosophila organisms, embryo lysates, and cells to characterize various loss-of-function phenotypes. Zamore and colleagues found that dsRNA added to Drosophila embryo lysates was processed to 21-23 nucleotide species. They also found that the homologous endogenous mRNA was cleaved only in the region corresponding to the introduced dsRNA and that cleavage occurred at 21-23 nucleotide intervals.

Current models of RNAi divide the process of inhibition into broad “initiation” and “effector” stages. In the initiation step, input dsRNA is digested into 21-23 nucleotide small interfering RNAs (siRNAs), which have also been called “guide RNAs.” Evidence indicates that siRNAs are produced when the enzyme Dicer, a member of the RNase III family of dsRNA-specific ribonucleases, processively cleaves dsRNA in an ATP-dependent, processive manner. Successive cleavage events degrade the RNA to 19-21 bp duplexes (siRNAs), each with 2-nucleotide 3′ overhangs. Inhibitory nucleic acids of the present disclosure can be enzymatically cleaved, for example in vivo, to produce siRNAs from 10 to about 30 nucleotides, typically about 19 to about 23 nucleotides.

In the effector step, the siRNA duplexes bind to a nuclease complex to form what is known as the RNA-induced silencing complex, or RISC. An ATP-depending unwinding of the siRNA duplex is required for activation of the RISC. The active RISC then targets the homologous transcript by base pairing interactions and cleaves the mRNA ˜12 nucleotides from the 3′ terminus of the siRNA. Although the mechanism of cleavage is at this date unclear, research indicates that each RISC contains a single siRNA and an RNase that appears to be distinct from Dicer. Because of the remarkable potency of RNAi in some organisms, an amplification step within the RNAi pathway has also been proposed. Amplification could occur by copying of the input dsRNAs, which would generate more siRNAs, or by replication of the siRNAs themselves. Alternatively or in addition, amplification could be effected by multiple turnover events of the RISC. One embodiment encompasses the in vivo amplification of the siRNAs disclosed herein. Additionally, the siRNAs described herein can form a complex with additional proteins and/or cofactors to enzymatically cleave a target mRNA.

2.4 Polynucleotide Compositions

One embodiment of the present disclosure provides a vector having a promoter operably linked to polynucleotide encoding a modified bone morphogenic polypeptide that binds to a bone morphogenic protein receptor without activating said receptor. For example, BMP4 can be modified to bind to BMP receptors on vascular cells without triggering signal transduction through the receptor. Fragments of BMP protein or BMP proteins having deletions or substitutions of amino acids can be screened for binding to vascular cell BMP receptors, and the signal transduction for the BMP receptor can be assayed, for example by determining the activation of NFκB induced gene expression. The nucleotide sequence corresponding to a fragment of BMP, for example BMP4, or an engineered BMP that does binds to BMP receptors without activating the receptors can then be cloned into an expression vector. The vascular cells can be contacted with the vector to express the modified BMP which can be expressed and secreted.

The engineering of DNA segment(s) for expression in a prokaryotic or eukaryotic system may be performed by techniques generally known to those of skill in recombinant expression. It is believed that virtually any expression system may be employed in the expression of the compositions disclosed herein.

As used herein, the terms “engineered” and “recombinant” cells are intended to refer to a cell into which an exogenous DNA segment or gene, such as a cDNA or gene has been introduced. Therefore, engineered cells are distinguishable from naturally occurring cells which do not contain a recombinantly introduced exogenous DNA segment or gene. Engineered cells are thus cells having a polynucleotide or genes introduced through the hand of man. Recombinant cells include those having an introduced cDNA or genomic DNA, and also include genes positioned adjacent to a promoter not naturally associated with the particular introduced gene.

To express a recombinant encoded protein or peptide, whether mutant or wild-type in accordance with the present disclosure one would prepare an expression vector that comprises one of the disclosed polynucleotides under the control of one or more promoters. To bring a coding sequence “under the control of” a promoter, one positions the 5′ end of the translational initiation site of the reading frame generally between about 1 and 50 nucleotides “downstream” of (i.e., 3′ of) the chosen promoter. The “upstream” promoter stimulates transcription of the inserted DNA and promotes expression of the encoded recombinant protein. This is the meaning of “recombinant expression” in the context used here.

Many standard techniques are available to construct expression vectors containing the appropriate nucleic acids and transcriptional/translational control sequences in order to achieve protein or peptide expression in a variety of host-expression systems. Cell types available for expression include, but are not limited to, bacteria, such as E. coli and B. subtilis transformed with recombinant phage DNA, plasmid DNA or cosmid DNA expression vectors.

Certain examples of prokaryotic hosts are E. coli strain RR1, E. coli LE392, E. coli B, E. coli chi. 1776 (ATCC No.31537) as well as E. coli W3110 (F-, lambda-, prototrophic, ATCC No. 273325); bacilli such as Bacillus subtilis; and other enterobacteriaceae such as Salmonella typhimurium, Serratia marcescens, and various Pseudomonas species.

In general, plasmid vectors containing replicon and control sequences that are derived from species compatible with the host cell are used in connection with these hosts. The vector ordinarily carries a replication site, as well as marking sequences that are capable of providing phenotypic selection in transformed cells. For example, E. coli is often transformed using pBR322, a plasmid derived from an E. coli species. Plasmid pBR322 contains genes for ampicillin and tetracycline resistance and thus provides easy means for identifying transformed cells. The pBR322 plasmid, or other microbial plasmid or phage must also contain, or be modified to contain, promoters that can be used by the microbial organism for expression of its own proteins.

In addition, phage vectors containing replicon and control sequences that are compatible with the host microorganism can be used as transforming vectors in connection with these hosts. For example, the phage lambda may be utilized in making a recombinant phage vector that can be used to transform host cells, such as E. coli LE392.

Further useful vectors include pIN vectors; and pGEX vectors, for use in generating glutathione S-transferase (GST) soluble fusion proteins for later purification and separation or cleavage. Other suitable fusion proteins are those with β-galactosidase, ubiquitin, or the like.

Promoters that are most commonly used in recombinant DNA construction include the β-lactamase (penicillinase), lactose and tryptophan (trp) promoter systems. While these are the most commonly used, other microbial promoters have been discovered and utilized, and details concerning their nucleotide sequences have been published, enabling those of skill in the art to ligate them functionally with plasmid vectors.

For expression in Saccharomyces, the plasmid YRp7, for example, is commonly used. This plasmid contains the trp1 gene, which provides a selection marker for a mutant strain of yeast lacking the ability to grow in tryptophan, for example ATCC No. 44076 or PEP4-1. The presence of the trp1 lesion as a characteristic of the yeast host cell genome then provides an effective environment for detecting transformation by growth in the absence of tryptophan.

Suitable promoting sequences in yeast vectors include the promoters for 3-phosphoglycerate kinase or other glycolytic enzymes, such as enolase, glyceraldehyde-3-phosphate dehydrogenase, hexokinase, pyruvate decarboxylase, phosphofructokinase, glucose-6-phosphate isomerase, 3-phosphoglycerate mutase, pyruvate kinase, triosephosphate isomerase, phosphoglucose isomerase, and glucokinase. In constructing suitable expression plasmids, the termination sequences associated with these genes are also ligated into the expression vector 3′ of the sequence desired to be expressed to provide polyadenylation of the mRNA and termination.

Other suitable promoters, which have the additional advantage of transcription controlled by growth conditions, include the promoter region for alcohol dehydrogenase 2, isocytochrome C, acid phosphatase, degradative enzymes associated with nitrogen metabolism, and the aforementioned glyceraldehyde-3-phosphate dehydrogenase, and enzymes responsible for maltose and galactose utilization.

In addition to micro-organisms, cultures of cells derived from multicellular organisms may also be used as hosts. In principle, any such cell culture is workable, whether from vertebrate or invertebrate culture. In addition to mammalian cells, these include insect cell systems infected with recombinant virus expression vectors (e.g., baculovirus); and plant cell systems infected with recombinant virus expression vectors (e.g., cauliflower mosaic virus, CaMV; tobacco mosaic virus, TMV) or transformed with recombinant plasmid expression vectors (e.g., Ti plasmid) containing one or more coding sequences.

In a useful insect system, Autograph californica nuclear polyhidrosis virus (AcNPV) is used as a vector to express foreign genes. The virus grows in Spodoptera frugiperda cells. The isolated nucleic acid coding sequences are cloned into non-essential regions (for example the polyhedron gene) of the virus and placed under control of an AcNPV promoter (for example, the polyhedron promoter). Successful insertion of the coding sequences results in the inactivation of the polyhedron gene and production of non-occluded recombinant virus (i.e., virus lacking the proteinaceous coat coded for by the polyhedron gene). These recombinant viruses are then used to infect Spodoptera frugiperda cells in which the inserted gene is expressed (e.g., U.S. Pat. No. 4,215,051).

Examples of useful mammalian host cell lines are VERO and HeLa cells, Chinese hamster ovary (CHO) cell lines, W138, BHK, COS-7, 293, HepG2, NIH3T3, RIN and MDCK cell lines. In addition, a host cell may be chosen that modulates the expression of the inserted sequences, or modifies and processes the gene product in the specific fashion desired. Such modifications (e.g., glycosylation) and processing (e.g., cleavage) of protein products may be important for the function of the encoded protein.

Different host cells have characteristic and specific mechanisms for the post-translational processing and modification of proteins. Appropriate cell lines or host systems can be chosen to ensure the correct modification and processing of the foreign protein expressed. Expression vectors for use in mammalian cells ordinarily include an origin of replication (as necessary), a promoter located in front of the gene to be expressed, along with any necessary ribosome binding sites, RNA splice sites, polyadenylation site, and transcriptional terminator sequences. The origin of replication may be provided either by construction of the vector to include an exogenous origin, such as may be derived from SV40 or other viral (e.g., Polyoma, Adeno, VSV, BPV) source, or may be provided by the host cell chromosomal replication mechanism. If the vector is integrated into the host cell chromosome, the latter is often sufficient.

The promoters may be derived from the genome of mammalian cells (e.g., metallothionein promoter) or from mammalian viruses (e.g., the adenovirus late promoter; the vaccinia virus 7.5K promoter). Further, it is also possible, and may be desirable, to utilize promoter or control sequences normally associated with the desired gene sequence, provided such control sequences are compatible with the host cell systems.

A number of viral based expression systems may be utilized, for example, commonly used promoters are derived from polyoma, Adenovirus 2, cytomegalovirus and Simian Virus 40 (SV40). The early and late promoters of SV40 virus are useful because both are obtained easily from the virus as a fragment which also contains the SV40 viral origin of replication. Smaller or larger SV40 fragments may also be used, provided there is included the approximately 250 bp sequence extending from the HinDIII site toward the BglI site located in the viral origin of replication.

In cases where an adenovirus is used as an expression vector, the coding sequences may be ligated to an adenovirus transcription/translatio- n control complex, e.g., the late promoter and tripartite leader sequence. This chimeric gene may then be inserted in the adenovirus genome by in vitro or in vivo recombination. Insertion in a non-essential region of the viral genome (e.g., region E1 or E3) will result in a recombinant virus that is viable and capable of expressing proteins in infected hosts.

Specific initiation signals may also be required for efficient translation of the claimed isolated nucleic acid coding sequences. These signals include the ATG initiation codon and adjacent sequences. Exogenous translational control signals, including the ATG initiation codon, may additionally need to be provided. One of ordinary skill in the art would readily be capable of determining this need and providing the necessary signals. It is well known that the initiation codon must be in-frame (or in-phase) with the reading frame of the desired coding sequence to ensure translation of the entire insert. These exogenous translational control signals and initiation codons can be of a variety of origins, both natural and synthetic. The efficiency of expression may be enhanced by the inclusion of appropriate transcription enhancer elements or transcription terminators.

In eukaryotic expression, one will also typically desire to incorporate into the transcriptional unit an appropriate polyadenylation site (e.g., 5′-AATAAA-3′, SEQ ID NO:) if one was not contained within the original cloned segment. Typically, the poly A addition site is placed about 30 to 2000 nucleotides “downstream” of the termination site of the protein at a position prior to transcription termination.

For long-term, high-yield production of recombinant proteins, stable expression is preferred. For example, cell lines that stably express constructs encoding proteins may be engineered. Rather than using expression vectors that contain viral origins of replication, host cells can be transformed with vectors controlled by appropriate expression control elements (e.g., promoter, enhancer, sequences, transcription terminators, polyadenylation sites, etc.), and a selectable marker. Following the introduction of foreign DNA, engineered cells may be allowed to grow for 1-2 days in an enriched medium, and then are switched to a selective medium. The selectable marker in the recombinant plasmid confers resistance to the selection and allows cells to stably integrate the plasmid into their chromosomes and grow to form foci, which in turn can be cloned and expanded into cell lines.

A number of selection systems may be used, including, but not limited, to the herpes simplex virus thymidine kinase, hypoxanthine-guanine and adenine phosphoribosyltransferase genes, in tk⁻, hgprt⁻ or aprt⁻ cells, respectively. Also, antimetabolite resistance can be used as the basis of selection for dhfr, which confers resistance to methotrexate; gpt, which confers resistance to mycophenolic acid; neo, which confers resistance to the aminoglycoside G-418; and hygro, which confers resistance to hygromycin.

It is contemplated that the isolated polynucleotides of the disclosure may be “overexpressed”, i.e., expressed in increased levels relative to its natural expression in human cells, or even relative to the expression of other proteins in the recombinant host cell. Such overexpression may be assessed by a variety of methods, including radio-labeling and/or protein purification. However, simple and direct methods are preferred, for example, those involving SDS/PAGE and protein staining or western blotting, followed by quantitative analyses, such as densitometric scanning of the resultant gel or blot. A specific increase in the level of the recombinant protein or peptide in comparison to the level in natural human cells is indicative of overexpression, as is a relative abundance of the specific protein in relation to the other proteins produced by the host cell and, e.g., visible on a gel.

Further aspects of the present disclosure concern the purification, and in particular embodiments, the substantial purification, of an encoded protein or peptide. The term “purified protein or peptide” as used herein, is intended to refer to a composition, isolatable from other components, wherein the protein or peptide is purified to any degree relative to its naturally-obtainable state. A purified protein or peptide therefore also refers to a protein or peptide, free from the environment in which it may naturally occur.

Generally, “purified” will refer to a protein or peptide composition that has been subjected to fractionation to remove various other components, and which composition substantially retains its expressed biological activity. Where the term “substantially purified” is used, this designation will refer to a composition in which the protein or peptide forms the major component of the composition, such as constituting about 50% or more of the proteins in the composition.

Various methods for quantifying the degree of purification of the protein or peptide will be known to those of skill in the art in light of the present disclosure. These include, for example, determining the specific activity of an active fraction, or assessing the number of polypeptides within a fraction by SDS/PAGE analysis. A preferred method for assessing the purity of a fraction is to calculate the specific activity of the fraction, to compare it to the specific activity of the initial extract, and to thus calculate the degree of purity, herein assessed by a “-fold purification number”. The actual units used to represent the amount of activity will, of course, be dependent upon the particular assay technique chosen to follow the purification and whether or not the expressed protein or peptide exhibits a detectable activity.

Various techniques suitable for use in protein purification will be well known to those of skill in the art. These include, for example, precipitation with ammonium sulphate, polyethylene glycol, antibodies and the like or by heat denaturation, followed by centrifugation; chromatography steps such as ion exchange, gel filtration, reverse phase, hydroxylapatite and affinity chromatography; isoelectric focusing; gel electrophoresis; and combinations of such and other techniques. As is generally known in the art, it is believed that the order of conducting the various purification steps may be changed, or that certain steps may be omitted, and still result in a suitable method for the preparation of a substantially purified protein or peptide.

There is no general requirement that the protein or peptide always be provided in their most purified state. Indeed, it is contemplated that less substantially purified products will have utility in certain embodiments. Partial purification may be accomplished by using fewer purification steps in combination, or by utilizing different forms of the same general purification scheme. For example, it is appreciated that a cation-exchange column chromatography performed utilizing an HPLC apparatus will generally result in a greater-fold purification than the same technique utilizing a low pressure chromatography system. Methods exhibiting a lower degree of relative purification may have advantages in total recovery of protein product, or in maintaining the activity of an expressed protein.

It is known that the migration of a polypeptide can vary, sometimes significantly, with different conditions of SDS/PAGE (Capaldi et al., Biochem. Biophys. Res. Comm., 76:425, 1977). It will therefore be appreciated that under differing electrophoresis conditions, the apparent molecular weights of purified or partially purified expression products may vary.

For some embodiments, it will be desired to produce antibodies that bind with high specificity to the protein product(s) of an isolated nucleic acid selected from the group comprising the sequences disclosed herein or a variant thereof. Means for preparing and characterizing antibodies are well known in the art (See, e.g., Antibodies: A Laboratory Manual, Cold Spring Harbor Laboratory, 1988, incorporated herein by reference).

Methods for generating polyclonal antibodies are well known in the art. Briefly, a polyclonal antibody is prepared by immunizing an animal with an antigenic composition and collecting antisera from that immunized animal. A wide range of animal species can be used for the production of antisera. Typically the animal used for production of antisera is a rabbit, a mouse, a rat, a hamster, a guinea pig or a goat. Because of the relatively large blood volume of rabbits, a rabbit is a preferred choice for production of polyclonal antibodies.

As is well known in the art, a given composition may vary in its immunogenicity. It is often necessary therefore to boost the host immune system, as may be achieved by coupling a peptide or polypeptide immunogen to a carrier. Exemplary and preferred carriers are keyhole limpet hemocyanin (KLH) and bovine serum albumin (BSA). Other albumins such as ovalbumin, mouse serum albumin or rabbit serum albumin can also be used as carriers. Means for conjugating a polypeptide to a carrier protein are well known in the art and include glutaraldehyde, m-maleimidobenzoyl-N-hy-droxysuccinimide ester, carbodiimide and bis-biazotized benzidine.

As is also well known in the art, the immunogenicity of a particular immunogen composition can be enhanced by the use of non-specific stimulators of the immune response, known as adjuvants. Exemplary and preferred adjuvants include complete Freund's adjuvant (a non-specific stimulator of the immune response containing killed Mycobacterium tuberculosis), incomplete Freund's adjuvants and aluminum hydroxide adjuvant.

The amount of immunogen composition used in the production of polyclonal antibodies varies upon the nature of the immunogen as well as the animal used for immunization. A variety of routes can be used to administer the immunogen (subcutaneous, intramuscular, intradermal, intravenous and intraperitoneal). The production of polyclonal antibodies may be monitored by sampling blood of the immunized animal at various points following immunization. A second, booster injection, may also be given. The process of boosting and titering is repeated until a suitable titer is achieved. When a desired level of immunogenicity is obtained, the immunized animal can be bled and the serum isolated and stored, and/or in some cases the animal can be used to generate monoclonal antibodies (MAbs). For production of rabbit polyclonal antibodies, the animal can be bled through an ear vein or alternatively by cardiac puncture. The removed blood is allowed to coagulate and then centrifuged to separate serum components from whole cells and blood clots. The serum may be used as is for various applications or the desired antibody fraction may be purified by well-known methods, such as affinity chromatography using another antibody or a peptide bound to a solid matrix.

Monoclonal antibodies (MAbs) may be readily prepared through use of well-known techniques, such as those exemplified in U.S. Pat. No. 4,196,265, incorporated herein by reference. Typically, this technique involves immunizing a suitable animal with a selected immunogen composition, e.g., a purified or partially purified expressed protein, polypeptide or peptide. The immunizing composition is administered in a manner that effectively stimulates antibody producing cells.

The methods for generating monoclonal antibodies (MAbs) generally begin along the same lines as those for preparing polyclonal antibodies. Rodents such as mice and rats are preferred animals, however, the use of rabbit, sheep or frog cells is also possible. The use of rats may provide certain advantages (Goding, 1986, pp. 60-61), but mice are preferred, with the BALB/c mouse being most preferred as this is most routinely used and generally gives a higher percentage of stable fusions.

The animals are injected with antigen as described above. The antigen may be coupled to carrier molecules such as keyhole limpet hemocyanin if necessary. The antigen would typically be mixed with adjuvant, such as Freund's complete or incomplete adjuvant. Booster injections with the same antigen would occur at approximately two-week intervals.

Following immunization, somatic cells with the potential for producing antibodies, specifically B lymphocytes (B cells), are selected for use in the MAb generating protocol. These cells may be obtained from biopsied spleens, tonsils or lymph nodes, or from a peripheral blood sample. Spleen cells and peripheral blood cells are preferred, the former because they are a rich source of antibody-producing cells that are in the dividing plasmablast stage, and the latter because peripheral blood is easily accessible. Often, a panel of animals will have been immunized and the spleen of animal with the highest antibody titer will be removed and the spleen lymphocytes obtained by homogenizing the spleen with a syringe. Typically, a spleen from an immunized mouse contains approximately 5'10⁷ to 2×10⁸ lymphocytes.

The antibody-producing B lymphocytes from the immunized animal are then fused with cells of an immortal myeloma cell, generally one of the same species as the animal that was immunized. Myeloma cell lines suited for use in hybridoma-producing fusion procedures preferably are non-antibody-producing, have high fusion efficiency, and have enzyme deficiencies that render them incapable of growing in certain selective media that support the growth of only the desired fused cells (hybridomas).

Any one of a number of myeloma cells may be used, as are known to those of skill in the art (Goding, 1986). For example, where the immunized animal is a mouse, one may use P3-X63/Ag8, X63-Ag8.653, NS1/1.Ag 4 1, Sp210-Ag14, FO, NSO/U, MPC-11, MPC11-X45-GTG 1.7 and S194/5XX0 Bul; for rats, one may use R210.RCY3, Y3-Ag 1.2.3, IR983F and 4B210; and U-266, GM1500-GRG2, LICR-LON-HMy2 and UC729-6 are all useful in connection with human cell fusions.

One preferred murine myeloma cell is the NS-1 myeloma cell line (also termed P3-NS-1-Ag4-1), which is readily available from the NIGMS Human Genetic Mutant Cell Repository by requesting cell line repository number GM3573. Another mouse myeloma cell line that may be used is the 8-azaguanine-resistant mouse murine myeloma SP2/0 non-producer cell line.

Methods for generating hybrids of antibody-producing spleen or lymph node cells and myeloma cells usually comprise mixing somatic cells with myeloma cells in a 2:1 proportion, though the proportion may vary from about 20:1 to about 1:1, respectively, in the presence of an agent-or agents (chemical or electrical) that promote the fusion of cell membranes. Fusion methods using Sendai virus have been described by Kohler and Milstein (1975; 1976), and those using polyethylene glycol (PEG), such as 37% (v/v) PEG, by Gefter et al. (1977). The use of electrically induced fusion methods is also appropriate (Goding, 1986).

Fusion procedures usually produce viable hybrids at low frequencies, about 1×10⁻⁶ to 1×10^{-8.} However, this low frequency does not pose a problem, as the viable, fused hybrids are differentiated from the parental, unfused cells (particularly the unfused myeloma cells that would normally continue to divide indefinitely) by culturing in a selective medium. The selective medium is generally one that contains an agent that blocks the de novo synthesis of nucleotides in the tissue culture media. Exemplary and preferred agents are aminopterin, methotrexate, and azaserine. Aminopterin and methotrexate block de novo synthesis of both purines and pyrimidines, whereas azaserine blocks only purine synthesis. Where aminopterin or methotrexate is used, the media is supplemented with hypoxanthine and thymidine as a source of nucleotides (HAT medium). Where azaserine is used, the media is supplemented with hypoxanthine.

The preferred selection medium is HAT. Only cells capable of operating nucleotide salvage pathways are able to survive in HAT medium. The myeloma cells are defective in key enzymes of the salvage pathway, e.g., hypoxanthine phosphoribosyl transferase (HPRT), and thus they cannot survive. The B cells can operate this pathway, but they have a limited life span in culture and generally die within about two weeks. Therefore, the only cells that can survive in the selective media are those hybrids formed from myeloma and B cells.

This culturing provides a population of hybridomas from which specific hybridomas are selected. Typically, selection of hybridomas is performed by culturing the cells by single-clone dilution in microtiter plates, followed by testing the individual clonal supernatants (after about two to three weeks) for the desired reactivity. The assay should be sensitive, simple and rapid, such as radioimmunoassays, enzyme immunoassays, cytotoxicity assays, plaque assays, dot immunobinding assays, and the like.

The selected hybridomas would then be serially diluted and cloned into individual antibody-producing cell lines, which can then be propagated indefinitely to provide MAbs. The cell lines may be exploited for MAb production in two basic ways. A sample of the hybridoma can be injected (often into the peritoneal cavity) into a histocompatible animal of the type that was used to provide the somatic and myeloma cells for the original fusion. The injected animal develops tumors secreting the specific monoclonal antibody produced by the fused cell hybrid. The body fluids of the animal, such as serum or ascites fluid, can then be tapped to provide MAbs in high concentration. The individual cell lines could also be cultured in vitro, where the MAbs are naturally secreted into the culture medium from which they can be readily obtained in high concentrations. MAbs produced by either means may be further purified, if desired, using filtration, centrifugation and various chromatographic methods such as HPLC or affinity chromatography.

Large amounts of the monoclonal antibodies of the present invention may also be obtained by multiplying hybridoma cells in vivo. Cell clones are injected into mammals that are histocompatible with the parent cells, e.g., syngeneic mice, to cause growth of antibody-producing tumors. Optionally, the animals are primed with a hydrocarbon, especially oils such as pristane (tetramethylpentadecane) prior to injection.

In accordance with the present invention, fragments of the monoclonal antibody of the present disclosure can be obtained from the monoclonal antibody produced as described above, by methods which include digestion with enzymes such as pepsin or papain and/or cleavage of disulfide bonds by chemical reduction. Alternatively, monoclonal antibody fragments encompassed by the present invention can be synthesized using an automated peptide synthesizer, or by expression of full-length gene or of gene fragments in E. coli.

The monoclonal conjugates of the present invention are prepared by methods known in the art, e.g., by reacting a monoclonal antibody prepared as described above with, for instance, an enzyme in the presence of a coupling agent such as glutaraldehyde or periodate. Conjugates with fluorescein markers are prepared in the presence of these coupling agents or by reaction with an isothiocyanate. Conjugates with metal chelates are similarly produced. Other moieties to which antibodies may be conjugated include radionuclides such as ³H, ¹²⁵I, ¹³¹I, ³²P, ³⁵S, ¹⁴C, ⁵¹Cr, ³⁶Cl, ⁵⁷Co, ⁵⁸Co, ⁵⁹Fe, ⁷⁵Se, and ¹⁵²Eu are other useful labels that can be conjugated to antibodies. Radioactively labeled monoclonal antibodies of the present invention are produced according to well-known methods in the art. For instance, monoclonal antibodies can be iodinated by contact with sodium or potassium iodide and a chemical oxidizing agent such as sodium hypochlorite, or an enzymatic oxidizing agent, such as lactoperoxidase. Monoclonal antibodies according to the invention may be labeled with technetium-⁹⁹ by ligand exchange process, for example, by reducing pertechnate with stannous solution, chelating the reduced technetium onto a Sephadex column and applying the antibody to this column or by direct labelling techniques, e.g., by incubating pertechnate, a reducing agent such as SnCl₂, a buffer solution such as sodium-potassium phthalate solution, and the antibody.

It will be appreciated by those of skill in the art that monoclonal or polyclonal antibodies specific for antagonists disclosed herein will have utilities in several types of applications that are within the scope of the present invention.

2.5 Methods

Other embodiments of the present disclosure provide methods of using the disclosed antagonists. One embodiment provides a method of decreasing or inhibiting monocyte adhesion to vascular cells by inhibiting binding of bone morphogenic polypeptide to the vascular cells. Inhibition is accomplished by contacting bone morphogenic polypeptide present in vascular fluid or tissue in contact with said vascular cells with a bone morphogenic polypeptide antagonist in an amount sufficient to inhibit or reduce the expression of cell adhesion molecules by said vascular cells. Exemplary antagonists are specific for BMP4 protein or fragment thereof. Because BMP4 is a secreted protein, BMP4 can be found in biologic fluid bathing vascular cells. Contacting the biologic fluid which includes, but is not limited to, interstitial fluid, blood, plasma, lymphatic fluid, cerebrospinal fluid, and saliva, with the disclosed antagonists of BMPs results in inactivating the BMPs for example, by forming complexes between the BMP and the antagonist. Such complexes cannot bind to BMP receptors including BMPR-I and BMPR-II. Alternatively, the antagonist complex can bind to BMP receptors but do not trigger the signal transduction pathway of the receptor. Inactive BMPs cannot participate in signal transduction pathways of vascular cells to activate NFκB regulated gene expression. Accordingly, cell adhesion molecules such as ICAM-1 are not upregulated or expressed in response to BMPs. Without cell adhesion molecules expressed on the surface of vascular cells, monocytes do not adhere to the vascular cells.

Another embodiment provides a method of inhibiting a vascular inflammatory response by contacting extracellular vascular fluid with an amount of bone morphogenic protein antagonist sufficient to inhibit binding of bone morphogenic protein to vascular cells in contact with said vascular fluid. Vascular inflammatory response includes the recruitment of monocytes to vascular cells by expressing cell adhesion molecules on the cell surface. By inactivating or inhibiting the binding of BMPs, for example BMP4, to vascular cells through the BMP receptors, the signal transduction pathway leading to increased expression of cell adhesion molecules such as ICAM-1 is inhibited. Inhibition of ICAM-1 expression inhibits the inflammatory response by reducing the ability of monocytes to bind to the vascular cells.

Still another embodiment provides a method of inhibiting vascular inflammation by contacting vascular cells with a bone morphogenic protein receptor antagonist in an amount sufficient to inhibit or reduce binding of bone morphogenic protein to said vascular cells. In this embodiment, BMP receptors, for example on vascular cells, are bound with an antagonist that does not trigger the BMP receptor signal transduction pathway. BMP receptors bound with the disclosed antagonists cannot bind functional BMPs, for example BMP4, and therefore, ICAM-1 expression is not increased through the BMP receptor signal transduction pathway. Yet another embodiment provides a method of inhibiting vascular inflammation by contacting vascular cells with an inhibitory polynucleotide specific for a bone morphogenic polypeptide, bone morphogenic protein receptor, or a combination thereof.

Other disclosed methods include a method for treating vascular inflammation by administering to a host an amount of bone morphogenic protein antagonist, bone morphogenic protein receptor antagonist, or a combination thereof sufficient to inhibit binding of bone morphogenic protein or a fragment thereof to vascular cells of the host. A host includes any organism, for example mammals such as humans. Administration of the antagonists interrupts, inhibits, or blocks the BMP receptor signal transduction pathway in vascular cells. This inhibition prevents, inhibits, or reduces the expression of cell adhesion molecules, for example ICAM-1, on the surface of vascular cells. In the absence of sufficient amounts of ICAM-1 for monocyte binding to vascular cells, vascular inflammation is reduced or inhibited. Specific aspects of inflammation that can be treated include, but are not limited to, atherosclerosis and or arteriosclerosis.

It will be appreciated by those of skill in the art that the disclosed compositions and methods are also useful for the treatment, identification, or prevention of pathologies having an inflammation basis or component including, but not limited to, atherosclerosis, rheumatoid arthritis, lupus, multiple sclerosis, diabetes, Sjogren's syndrome, ankylosing spondylitis, crohn's disease, inflammatory bowel disease, and psoriasis.

2.6 Pharmaceutical Compositions Exemplary embodiments of the present disclosure include pharmaceutical compositions that can be manufactured by processes well known in the art, e.g., by means of conventional mixing, dissolving, granulating, dragee-making, levigating, emulsifying, encapsulating, entrapping, lyophilizing processes or spray drying. Moreover, in certain embodiments, the compositions of the present disclosure may be formulated for horticultural or agricultural use. Such formulations include dips, sprays, seed dressings, stem injections, sprays, and mists. The ordinarily skilled clinician can determine and prescribe an effective amount of disclosed antagonist for a subject using the methods described herein. To do so, the physician or veterinarian may, for example, prescribe a relatively low dose at first, subsequently increasing the dose until an appropriate response is obtained. Other factors include the subject's age, body weight, general health, gender, and diet, the time of administration, the route of administration, the rate of excretion, other drugs used in combination. Representative dosages are typically 1 to 100 mg, more typically 5 to 50 mg, even more typically 10 to 20 mg.

The compositions of the present disclosure can be liquids or lyophilized or otherwise dried formulations and include diluents of various buffer content (e.g., Tris-HCl, acetate, phosphate), pH and ionic strength, additives such as albumin or gelatin to prevent absorption to surfaces, a surfactant such as a polysorbate surfactant (e.g., TWEEN 20, TWEEN 40, TWEEN 60, and TWEEN 80), a pheoxypolyethoxyethanol surfactant (e.g., TRITON X-100, X-301, X-165, X-102, and X-200, and TYLOXAPOL) Pluronic F68, or sodium dodecyl sulfate, solubilizing agents (e.g., glycerol, polyethylene glycerol), anti-oxidants (e.g., ascorbic acid, sodium metabisulfite), preservatives (e.g., Thimerosal, benzyl alcohol, parabens), bulking substances or tonicity modifiers (e.g., lactose, mannitol), covalent attachment of polymers such as polyethylene glycol to the protein, complexation with metal ions, or incorporation of the material into or onto particulate preparations of polymeric compounds such as polylactic acid, polglycolic acid, hydrogels, etc, or onto liposomes, microemulsions, micelles, unilamellar or multilamellar vesicles, erythrocyte ghosts, or spheroplasts. Such compositions will influence the physical state, solubility, stability, rate of in vivo release, and rate of in vivo clearance, controlled or sustained release compositions include formulation in lipophilic depots (e.g., fatty acids, waxes, oils).

The present disclosure contemplates formulations that may be employed in pharmaceutical and therapeutic compositions and applications suitable for inhibiting, interfering with, or reducing bone morphogenic protein signal transduction, for example in vascular cells. Such compositions may be employed to inhibit expression of proteins related to BMP signal transduction, interfere with signal transduction mechanisms of the BMP signal transduction, interfere with the formation, dissemination of BMPs, in particular BMP4, or reduce the adhesion of monocytes to vascular tissue.

For in vivo applications, the compositions can be administered in any effective pharmaceutically acceptable form to a host, for example warm blooded animals, including human and animal subjects. Generally, this entails preparing compositions that are essentially free of pyrogens, as well as other impurities that could be harmful to humans or animals.

Other embodiments of the disclosure provide particulate compositions coated with polymers (e.g., poloxamers or poloxamines). Still other embodiments of the compositions of the disclosure incorporate particulate forms protective coatings, protease inhibitors or permeation enhancers for various routes of administration, including parenteral, pulmonary, nasal and oral. In one embodiment the pharmaceutical composition is administered buccally, rectally, vaginally, topically, nasally, parenterally, paracancerally, transmucosally, transdermally, intramuscularly, intravenously, intradermally, subcutaneously, intraperitonealy, intraventricularly, intracranially, intratumorally, spray or in any other form effective to deliver active compositions.

For topical applications, the pharmaceutically acceptable carrier may take the form of a liquid, cream, foam, lotion, or gel, and may additionally comprise organic solvents, emulsifiers, gelling agents, moisturizers, stabilizers, surfactants, wetting agents, preservatives, time release agents, and minor amounts of humectants, sequestering agents, dyes, perfumes, and other components commonly employed in pharmaceutical compositions for topical administration.

Further, as used herein “pharmaceutically acceptable carrier” are well known to those skilled in the art and include, but are not limited to, 0.01-0.1M and preferably 0.05M phosphate buffer or 0.8% saline. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media.

Parenteral vehicles include sodium chloride solution, Ringer's dextrose, dextrose and sodium chloride, lactated Ringer's or fixed oils. Intravenous vehicles include fluid and nutrient replenishers, electrolyte replenishers such as those based on Ringer's dextrose, and the like. Preservatives and other additives may also be present, such as, for example, antimicrobials, antioxidants, collating agents, inert gases and the like.

Controlled or sustained release compositions include formulation in lipophilic depots (e.g., fatty acids, waxes, oils). Also comprehended by the disclosure are particulate compositions coated with polymers (e.g. poloxamers or poloxamines) and the compound coupled to antibodies directed against tissue-specific receptors, ligands or antigens or coupled to ligands of tissue-specific receptors.

Tablet and dosage forms of the compositions in which the emulsions are formulated for oral or topical administration include liquid capsules, and suppositories. In solid dosage forms for oral administration, the compositions may be admixed with one or more substantially inert diluent (e.g., sucrose, lactose, or starch, and the like) and may additionally comprise lubricating agents, buffering agents, enteric coatings, and other components well known to those skilled in the art.

Compounds modified by the covalent attachment of water-soluble polymers such as polyethylene glycol, copolymers of polyethylene glycol and polypropylene glycol, carboxymethyl cellulose, dextran, polyvinyl alcohol, polyvinylpyrrolidone or polyproline are known to exhibit substantially longer half-lives in blood following intravenous injection than do the corresponding unmodified compounds. Such modifications may also increase the compound's solubility in aqueous solution, eliminate aggregation, enhance the physical and chemical stability of the compound, and greatly reduce the immunogenicity and reactivity of the compound. As a result, the desired in vivo biological activity may be achieved by the administration of such polymer-compound abducts less frequently or in lower doses than with the unmodified compound.

In yet another embodiment, the pharmaceutical composition can be delivered in a controlled release system. For example, the agent may be administered using intravenous infusion, an implantable osmotic pump, a transdermal patch, liposomes, or other modes of administration. In one embodiment, a pump may be used ( Sefton (1987). CRC Crit. Ref: Biomed. Eng. 14:201; Buchwald et al. (1980). Surgery 88:507; Saudek et al. (1989). N. Engl. J. Med. 321:574). In another embodiment, polymeric materials can be used. In yet another embodiment a controlled release system can be placed in proximity of the therapeutic target, i.e., the brain, thus requiring only a fraction of the systemic. Preferably, a controlled release device is introduced into a subject in proximity of the site of inappropriate immune activation or a tumor. Other controlled release systems are discussed in the review by Langer (1990). Science 249:1527-1533.

In other embodiments, the compositions may be impregnated into absorptive materials, such as sutures, bandages, and gauze, or coated onto the surface of solid phase materials, such as surgical staples, zippers and catheters to deliver the compositions to a site for the prevention of microbial infection. Other delivery systems of this type will be readily apparent to those skilled in the art.

Examples of suitable oily vehicles or solvents for use with the present disclosure are vegetable or animal oils such as sunflower oil or fish-liver oil. Preparations can be effected both as dry and as wet granules. For parenteral administration (subcutaneous, intravenous, intra-arterial, or intramuscular injection), the compositions or their. physiologically tolerated derivatives such as salts, esters, N-oxides, and the like are converted into a solution, suspension, or emulsion, if desired with the substances customary and suitable for this purpose, for example, solubilizers or other auxiliaries. Examples are: sterile liquids such as water and oils, with or without the addition of a surfactant and other pharmaceutically acceptable adjuvants. Illustrative oils are those of petroleum, animal, vegetable, or synthetic origin, for example, peanut oil, soybean oil, or mineral oil. In general, water, saline, aqueous dextrose and related sugar solutions, and glycols such as propylene glycols or polyethylene glycol are preferred liquid carriers, particularly for injectable solutions.

In addition, if desired, the composition can contain minor amounts of auxiliary substances such as wetting or emulsifying agents, pH buffering agents which enhance the effectiveness of the active ingredient.

An active component can be formulated into the composition as neutralized pharmaceutically acceptable salt forms. Pharmaceutically acceptable salts include the acid addition salts (formed with the free amino groups of the polypeptide or antibody molecule) and which are formed with inorganic acids such as, for example, hydrochloric or phosphoric acids, or such organic acids as acetic, oxalic, tartaric, mandelic, and the like. Salts formed from the free carboxyl groups can also be derived from inorganic bases such as, for example, sodium, potassium, ammonium, calcium, or ferric hydroxides, and such organic bases as isopropylamine, trimethylamine, 2-ethylamino ethanol, histidine, procaine, and the like.

For topical administration to body surfaces using, for example, creams, gels, drops, and the like, the inhibitory nucleic acids and there prodrugs or their physiologically tolerated derivatives such as salts, esters, N-oxides, and the like are prepared and applied as solutions, suspensions, or emulsions in a physiologically acceptable diluent with or without a pharmaceutical carrier.

In another embodiment, the active compound can be delivered in a vesicle, in particular a liposome (see Langer (1990). Science, 249:1527-1533; Treat et al. (1989). in Lopez-Berestein and Fidler (eds.), Liposomes in the Therapy of Infectious Disease and Cancer, Liss, N.Y., pp. 353-365).

Suitable salts of the compositions disclosed herein include pharmaceutically acceptable salts. Other salts may, however, be useful in the preparation of the compounds according to the disclosure or of their pharmaceutically acceptable salts. Suitable pharmaceutically acceptable salts of the compounds of this disclosure include acid addition salts which may, for example, be formed by mixing a solution of the compound according to the disclosure with a solution of a pharmaceutically acceptable acid such as hydrochloric acid, sulphuric acid, methanesulphonic acids fumaric acid, maleic acid, succinic acid, acetic acid, benzoic: acid, oxalic acid, citric acid, tartaric acid, carbonic acid or phosphoric acid.

2.7 Combination Therapy

It is also an aspect of this disclosure that a composition described herein, or its prodrug, salt, or derivative might be combined with other therapeutic agents for the treatment of the diseases and disorders discussed above. Exemplary second therapeutic agents include but are not limited to anti-inflammatory agents, antioxidants, and cholesterol lowering agents. Suitable anti-inflammatory agents include steroid and non-steroid anti-inflammatory agents. Exemplary steroid anti-inflammatory agents include glucocorticoids such as dexamethasone, prednisolone. Additionally, non-steroid anti-inflammatory agents can be used such as probucol or esters thereof, ibuprophen, naprosyn, and the like. Representative antioxidants include, but are not limited to N-acetyl cysteine, vitamins A, C, or E, alpha lipoic acid, coenzyme Q10, and lycopene. Exemplary cholesterol lowering agents include, but are not limited to, HMG CoA reductase inhibitors, e.g., lovastatin, pravastatin and simvastatin; bile acid sequestrants, e.g., cholestyramine and colestipol and nicotinic acid (niacin); and gemfibrozil, probucol and clofibrate.

Other features of the invention will become apparent in the course of the following descriptions of exemplary embodiments which are given for illustration of the invention and are not intended to be limiting thereof.

### EXAMPLES

Materials and Methods

Endothelial cells: Mouse aortic endothelial cells (MAEC) were cultured and used at passages 4-8 as described (Cai, H., et al. (2002) J Bio Chem 107, 48311-48317). Human aortic endothelial cells (HAEC) purchased from Clonetics were cultured using EGM-2 bullet kit (Clonetics) and used at passages 4-8. Alternatively, endothelial cells obtained from the thoracic aortas of C57/BL6 control (MAE-wt) and p47^{phox}-null mice (MAE-p47^{-/-}) were isolated and cultured as described previously (Hwang, J. et al. (2003) J Biol Chem 278:47291-8), and used between passages 4-10.

Shear stress studies: Confluent endothelial monolayers grown in 100 mm tissue culture dishes were exposed to an arterial level of uni-directional LS (15 dyn/cm²) in the growth medium by rotating a Teflon cone (0.5° cone angle) as described previously. To mimic unstable shear conditions in vivo, endothelial cells were exposed to OS with directional changes of flow at 1 Hz cycle (±5 dyn/cm²) by rotating the cone back-and-forth using a stepping motor (Servo Motor) and a computer program (DC Motor Company, Atlanta, Ga.). In some studies, 5 dyn/cm² unidirectional LS was used for comparison to OS (±5 dyn/cm²).

Preparation of cell lysates and immunoblotting: Following experimental treatments, endothelial cell lysates were prepared and analyzed by Western blot analysis as described by us (Boo, Y. C., et al. (2002) Am J Physiol Heart Circ Physio! 283, H1819-1828; Boo, Y. C, et al. (2002) J Biol Chem 277, 3388-3396). Briefly, cells were washed in ice-cold phosphate buffered saline (PBS) and lysed in 0.1 ml boiling lysis buffer A (10 mM Tris-HCl, pH 7.6, 1 mM sodium vanadate and 1% SDS). The lysate was further homogenized by repeated aspiration through a 25-gauge needle. Protein content of each sample was measured by using a Bio-Rad DC assay (Jo, H., et al. (1997) J Biol Chem 272,1395-1401). To detect secreted BMP4 in conditioned media, endothelial monolayers were first washed in serum free DMEM supplemented with minimum non-essential amino acids and pyruvic acid, and exposed to OS, LS or static conditions for 1 day. The conditioned media were then centrifuged at 1,000×g for 10 min. Aliquots (2 ml) of the supernatant were collected and placed on ice with 10 ml ice cold acetone to precipitate protein for 30 minutes. Samples were pelleted by centrifugation (15,000×g for 10 min) and resuspended in 100 μl sample buffer for SDS-PAGE (Boo, Y. C., et al. (2002) Am J Physiol Heart Circ Physio! 283, H1819-1828; Boo, Y. C, et al. (2002) J Biol Chem 277, 3388-3396). Aliquots of cell lysates (20 μg protein each) were resolved on a 10% SDS-PAGE gel and transferred to a polyvinylidene difluoride membrane (Millipore). The membrane was incubated with a primary antibody overnight at 4° C., and then with a secondary antibody conjugated with alkaline phosphatase (1 hr at room temperature), which were detected by a chemiluminescence method (Jo, H., et al. (1997) J Biol Chem 272,1395-1401). The intensities of immunoreactive bands in Western blots were analyzed by using the NIH Image program. The following primary antibodies were used: a monoclonal BMP4 antibody, rabbit ICAM-1 antibody, goat VCAM1, and goat actin antibody (Santa Cruz).

DNA microarray analysis: DNA microarray analyses were performed with Affymetrix murine gene chip containing 12,000 genes (U74Av2, Affymetrix) and Motorola murine genome chip containing 10,000 genes (Motorola) according to the protocols provided by each manufacturer (Napoli, C., et al. (2002) Circulation 105, 1360-1367). Affymetrix chips were scanned and analyzed at the DNA Core facility at Emory University School of Medicine, while studies with Motorola chips, the entire process from reverse transcription to hybridization, scanning to initial data analysis was performed by using the manufacturer's protocol and laboratory (Chicago, Ill.).

Quantitative real-time polymerase chain reaction (PCR): Real time PCR for BMP4 was carried out as previously described (Sorescu, D., et al. (2002) Circulation 105, 1429-1435). Briefly, 4 μg of total RNA was reverse transcribed by using random primers and a Superscript-II kit (Life Technology) to synthesize first-strand cDNA- The cDNA was purified using a microbiospin 30 column (Bio-Rad) in Tris Buffer and stored at −20° C. until used. The cDNA was amplified using a LightCycler (Roche) RT-PCR machine. The mRNA copy numbers were determined based on standard curves generated with murine BMP4 and 18S templates. The 18S primers (50 nM at 61° C. annealing temperature, Ambion) were used as an internal control for real-time PCR using a LightCycler and capillaries (Roche), recombinant Taq polymerase (Life Technology), and Taq start antibody (Clontech). A quantitative RT-PCR using BMP4 primer pair (forward: CTGCGGGACTTCGAGGCGACACTTCT (SEQ ID NO: 11), reverse: TCTTCCTCCTCCTCCTCCCCAGACTG (SEQ ID NO: 12)) using endothelial RNA sample yielded a 130 base pair fragment on agarose gel electrophoresis. This pair of primer was verified by a nested PCR using other BMP4 primer pairs (forward: ATGGACTGTTATTATGCCTTGTTTTCTGTCAACACCATGATTC (SEQ ID NO: 13), reverse: CCACGTATAGTGAATGGCGACGGCAGTTCTT (SEQ ID NO: 14) and the pair-forward: GTCAACACCATGATTCCTGGTAACCGAATGCTGA (SEQ ID NO: 15), reverse: TTATACGGTGGAAGCCCTGTTCCCAGTCAG (SEQ ID NO: 16)) and by running DNA gels. Real-time PCR for BMP4 was carried out using the annealing temperature 65° C. and extension time for 7 sec in the PCR buffer (20 mM Tris Cl, pH 8.4 at 25° C., 50 mM KCl to which 250 μg/mL BSA, 200 μM deoxynucleotides) containing SYBR green (1:84,000 dilution), 0.05 U/μl Taq DNA polymerase and TaqStart antibody (1:100 dilution).

Fluorescence activated cytometry sorting (FACS) analysis: Treated cells were dissociated into single cell suspensions using 0.25% trypsin-EDTA and resuspended in a FACS buffer (Hank's buffered solution containing 5% FBS). Aliquots of cell suspensions were incubated with ICAM-1 antibody (R&D Systems) for 20 min on ice, washed twice with FACS buffer, and incubated with secondary antibody (fluorescein-5-isothiocyanate- or phycoerytherin-conjugated, Chemicon) for 20 min on ice in the dark. Then samples were washed again, fixed in 1% paraformaldehyde and analyzed by FACS (Calibur, Becton-Dickinson) using CellQuest software. The fluorescence intensity of ICAM-1 and forward cell scattering of 30,000 cells were measured and the geometric means calculated from histograms were shown. In some studies, HAEC were transfected with either BMP4 cloned in a bi-cistronic pAdTrack CMV vector or an empty vector, both expressing green fluorescent protein (GFP), using Lipofectamine 2000. In these experiments, ICAM-1 expression was measured in the red phycoerytherin channel, while the green channel was used to monitor the OFF expression-GFP expression was determined by FACS analysis and fluorescence microscopy (20-30% transfection efficiency). Because expression of GFP was similar among different treatment groups within the same experiment, it was unnecessary to normalize ICAM-1 expression data to GFP level. Human recombinant noggin was purchased from R&D Systems and used in all experiments at 50 ng/ml (Dale, L., and Jones, C. M. (1999) Bioessays 21, 751-760; Zimmerman, L. B., et al. (1996) Cell 86, 599-606; Brunei, L. J., et al. (1998) Science 280, 1455-1457).

Immunohistocheraical study: Frozen sections of the human coronary arteries obtained from patients undergoing heart transplantations were prepared (Sorescu, D., et al. (2002) Circulation 105, 1429-1435) and stained with antibodies specific for BMP4 (1:1,000 dilution, goat antibody, Santa Cruz), ICAM-1 (1:50 dilution, mouse antibody, R&D) or von Willebrand Factor (1:100 dilution, mouse antibody, Dako) for 2 hrs at room temperature, washed and followed by incubation with secondary antibodies (anti-goat IgG or anti-mouse IgG)-conjugated to alkaline phosphatase. Then the slides were washed and developed with a DAKO kit (DAKO). Photographs of the slides were taken using a Zeiss microscope. Fifteen different human coronary arteries from 6 different patients were examined containing various stages of atherosclerosis (from minimally diseased to fatty streak to advanced atheroma stage).

Monocyte Adhesion: Monocyte binding was determined under no-flow conditions using THP-1 monocytes (ATCC) by the method described by Chappel et al (Chappell, D. C., et al. (1998) Circ Res 82, 532-539). Briefly, THP-1 cells (5×10⁵ cells/ml) were labeled with a fluorescent dye 2′,7′-bis(carboxyethyl)-5(6)-carboxyfluorescein-AM (BCECF, Molecular Probes) (1 mg/ml) in serum-free RPMI medium for 45 min at 37° C. Following exposure to shear stress or BMP treatments in the presence or absence of noggin or vehicle, the endothelial cells were washed in RPMI medium before adding BCECF-loaded THP-1 cells (1:1 ratio). After a 30 min incubation at 37° C. under no-flow conditions, unbound monocytes were removed by washing the endothelial dishes 5 times with Hank's PBS. Bound monocytes were quantified by either counting the cells under a fluorescent microscope or by measuring the fluorescent intensity of cell lysates by fluorescence spectrophotometry using a plate reader. Both assays showed similar results. Some studies were performed with MAEC pretreated with 5 μg/ml of mouse-ICAM1 antibody (YN1, Southern Biolechonology) (Kevil, C. G., et al. (2001) Am J Physiol Cell Physiol 281, C1442-1447).

NF-κB assay: NF-κB activity was determined by using a NF-κB reporter construct, NF-κB-SEAP vector (lug, Clontech) expressing a secreted form of placental alkaline phosphatase driven by 4 KB sequences in tandem. This construct was co-transfected with 0.5 μg of either pAdTrack BMP4 or empty vector control using Lipofectamine 2000. Six hours post transfection, conditioned media were centrifuged, heat-treated at 65° C. (to inactivate endogenous alkaline phosphatase) for 30 min, and followed by chemiluminescence alkaline phosphatase assay according to the manufacturer's instructions.

Transfection of p47phox: In some studies MAE-p47^{-/-} cells were trasnfected withp47phox expression vector or an empty vector as described (Hwang, J. et al. (2003) J Biol Chem278-47291-8).

H₂O₂ assay: Cells grown in 6-well plates were treated with BMP4 or vehicle control for 18 hrs in the presence or absence of cell-permeable polyethylene glycol (PEG)-catalase (100 U/ml). Cells were then washed once with Hepes buffered saline solution (HBSS) followed by incubation with 7.5 μg/ml 2′, 7′-dichlorofluorescein diacetate (DCF-DA, Molecular Probes) in HBSS for 1 hr at 37 C° in the dark. Cells were washed with HBSS, incubated further for an additional 40 min in HBSS, and the DCF fluorescence intensities were measured in a Cytofluor Multi-Well plate reader (excitation at 475 nm and emission at 525 nm).

O₂⁻ Assay: O₂⁻ production was determined by electron spin resonance spectroscopy using methoxycarbonyl-2,2,5,5-tetramethyl-pyrrolidine (CMH) as a spin probe in the presence or absence of PEG-superoxide dismutase (PEG-SOD; 50 U/ml) to quantify the SOD-inhibitable portions of CM.

Statistical Analysis: Statistical significance was assessed by Student's t test using Microcal Origin statistical package.

### Example 1

### Differential Regulation of BMP4 Gene by LS and OS in Endothelial Cells

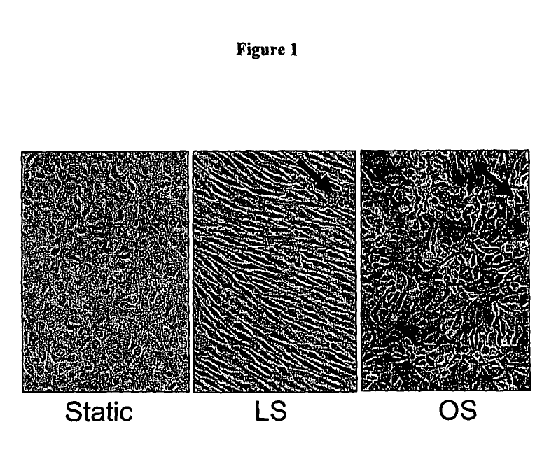

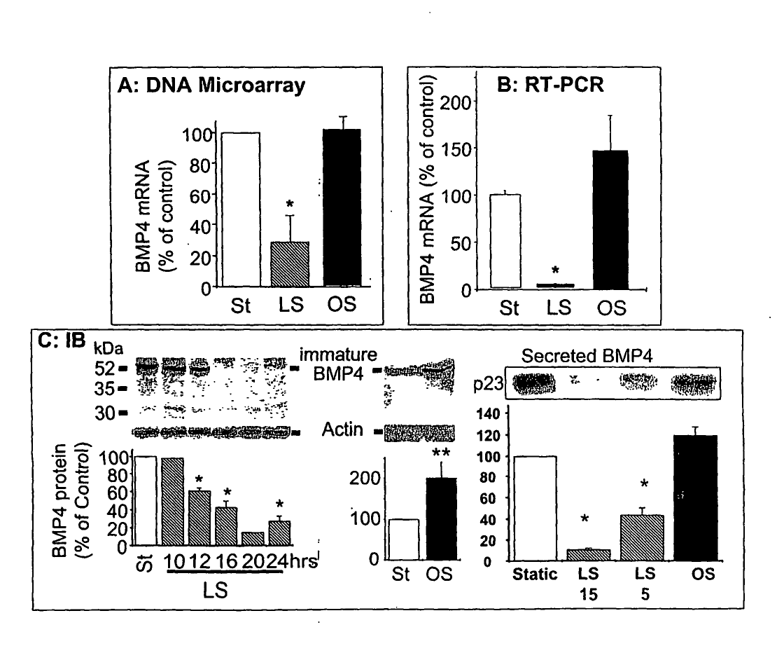

To identify the genes that may be responsible for the atheroprotective and pro-atherogenic effects of LS and OS, respectively, DNA microarray studies were performed using cultured MAEC. Exposing MAEC to LS, but not OS, for 1 day using the modified “cone-and-plate” device, induced a cell shape alignment to the direction of the flow from a typical polygonal “cobblestone shape” found in static cultured cells (FIG. 1). FIG. 1 shows confluent monolayers of MAEC were exposed to static condition (St), LS (15 dynes/cm²) or OS (±5 dyn/cm², 1 Hz cycle) for 24 his using the cone-and-plate apparatus. Following shear exposure, cell morphology was determined by light microscopy. Arrows indicate the direction of imposed shear stress.

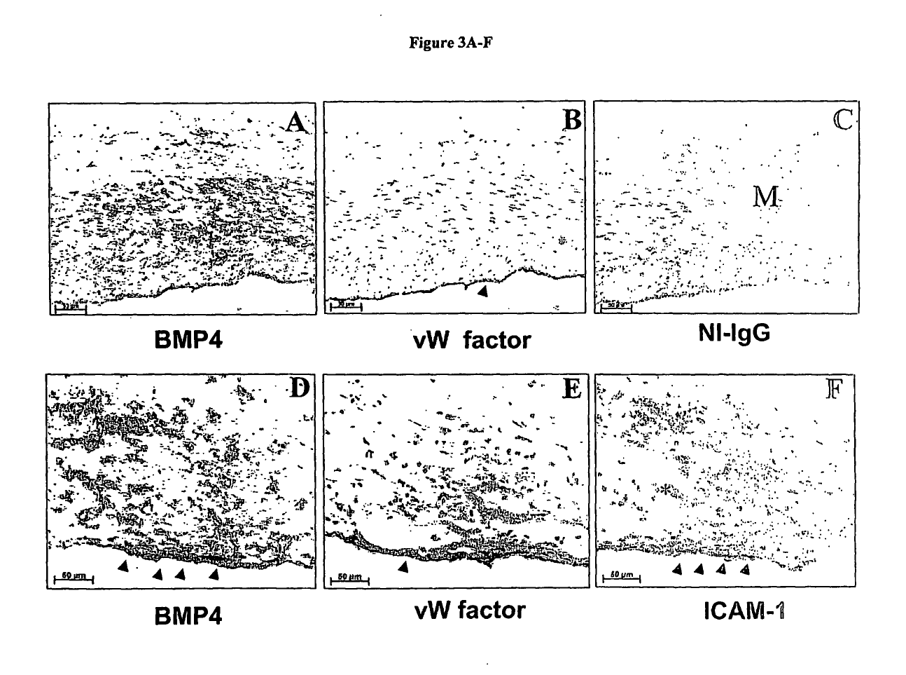

The total RNAs prepared from these cells were used to determine mRNA expression profiles by using Affymetrix and/or Motorola DNA chips according to the manufacturers′ protocols. The analyses of these studies showed that LS exposure significantly and consistently inhibited BMP4 mRNA level in MAEC by more than 60 to 80% of static control levels (FIG. 2A).

FIG. 2 shows MAEC exposed to static condition (St), LS or OS for 24 his (Panels A, B and C), except for a time-course study (C, left panel). In FIG. 2A total RNA prepared from each dish exposed to LS, OS or static condition was used in DNA microarray analysis using Afrymetrix murine chip (n=3 each for St and LS) or Motorola murine chip (n=3 each for St, LS and OS). Shown bar graph is the mean % level of BMP4±SEM using all data sets compared to that of static control values (* n=6, p<0.05).

Unlike LS, however, exposure of endothelial cells to OS did not inhibit BMP4 mRNA expression (FIG. 2A). We also found that LS exposure upregulated a well-known mechanosensitive gene, endothelial nitric oxide synthase (eNOS) mRNA level by more than 5-fold (5.6±1.2, n=3) above static controls. In addition, eNOS protein level was also increased two-fold above controls as determined by Western blot analysis using a monoclonal antibody (data not shown).

BMPs play an important role in bone formation, embryonic development and differentiation (Massague, J. (2000) Nat Rev Mol Cell Biol 1, 169-178; Hogan, B. L. (1996) Curr Opin Genetics Dev 6, 432-438). Although BMP4 protein has been found previously in calcified atherosclerotic plaques (Dhore, C. R., et al. (2001) Arterioscler Thromb Vasc Biol 21, 1998-2003), its expression and functional importance in endothelial cells have not been determined. Therefore, the microarray results were varified by independent methods at the levels of mRNA and protein as well as its functional roles in endothelial biology and pathobiology.

First, the BMP4 mRNA data was verified by using a quantitative real-time PCR method. Exposure of endothelial cells to LS almost eliminated the BMP4 mRNA level (n=6, p<0.001) (FIG. 2B). In FIG. 2B total RNAs obtained from above as well as known amounts of murine BMP4 standards were analyzed by real-time PCR. The BMP4 mRNA copy numbers were normalized against the 18S mRNA copy numbers. The bar graph shows the BMP4 mRNA levels expressed as % of static control values (mean±SEM, n=6 for St and LS and n=3 for OS) (*p<0.001). In contrast, OS marginally, statistically not significant, increased BMP4 mRNA level compared to that of static control (n=3). These results confirmed the DNA microarray results.

Next, BMP4 protein expression was determined by immunoblot studies. BMP4 protein is synthesized as an inactive precursor (48-55 kDa) that is proteolytically cleaved by proprotein convertases and the active ˜23 kDa protein is secreted (Massague, J. (2000) Nat Rev Mol Cell Biol 1, 169-178; Hogan, B. L. (1996) Curr Opin Genetics Dev 6, 432-438). In endothelial cell lysates, the BMP4 precursor was detected as a 54 kDa protein, and the mature form (p23) was detected in the conditioned media collected from static or shear exposed cells (FIG. 2C).

More particularly, in FIG. 2C cell lysates obtained from cells exposed to LS, OS or St were analyzed by Western blot with a BMP4 antibody or an actin antibody (used as a loading control). The band intensities were quantified and expressed as % of static controls as shown in the bar graphs. The left panel shows that LS significantly decreased BMP4 protein expression (*p<0.5, n=3-6, except for 20 hr group where n=2). The middle panel shows that OS significantly increased BMP4 expression (*p<0.5, n=6). In the right panel MAEC were exposed to 15 dyn/cm² of LS (LS 15 dynes), 5 dynes/cm² of LS or ±5 dynes/cm² of OS. After the shear, equal volumes of medium were precipitated and western blot performed with a BMP4 antibody. (* and ** p<0.5, n=3). Cell lysates (left and mid panels) and conditioned media (right panel) obtained from cells exposed to LS or OS were analyzed by Western blot with a BMP4 antibody or an actin antibody (used as a loading control). The band intensities were quantified and expressed as % of static controls (mean±SEM). Left panel: *p<0.5, n=3-6, except for 20hr group where n=2. Mid panel: *p<0.5, n=6. Right panel: * p<0.5.

Exposure of cells to LS significantly down-regulated expression of BMP4 precursor in a time-dependent manner (FIG. 2C). After 16 to 24 hours of LS exposure, BMP4 precursor expression was virtually undetectable (FIG. 2C, left panel, p<0.05). In contrast, exposure of MAEC to OS significantly increased BMP4 precursor protein level by 2-fold above control (FIG. 2C, middle panel, p<0.05). Consistent with the cell lysate result, the conditioned media of MAEC exposed to LS (15 dyn/cm²) showed a barely detectable amount of secreted form of BMP4 (p23) (FIG. 2C right panel). In contrast, OS exposure did not significantly change the p23 BMP4 level in the media, and decreased the levels of mature form found in the conditioned medium (FIG. 2C, right panel, p<0.05, t-test analysis). Since the cells were exposed to LS at 15 dyn/cm² and OS ±5 dyn/cm², the shear magnitude difference was evaluated as the potential source of the results observed so far. To address this question, the effects of LS and OS were compared using the same magnitudes (5 dyn/cm² LS vs. ±5 dyn/cm² OS). As shown as FIG. 2C (right panel), at the same shear magnitude, OS-exposed cells had more than 3-fold BMP4 protein than that of LS exposed cells. However, the higher LS magnitude (15 dyn/cm²) showed much lower amount of BMP4 than that of lower LS (5 dyn/cm2). These results show that LS exposure inhibits BMP4 expression in a force-dependent manner, while OS maintains high BMP4 expression.

### Example 2

### BMP4 Expression in the Selective Patches of Endothelial Cells Over Foam-Cell Lesions in Human Coronary Arteries

Next, the presence of BMP4 protein in endothelial cells of human atherosclerotic lesions from human coronary arteries was determined. The coronary arteries exhibiting a spectrum of atherosclerotic lesion complexity were obtained from patients undergoing heart transplantations and examined by immunohistochemical staining (Sorescu, D., et al. (2002) Circulation 105, 1429-1435). BMP4 protein expression was not apparent in the intimal endothelial cells in relatively normal, “minimally diseased” human coronary arteries (FIG. 3A) as well as advanced lesions (data not shown).

FIG. 3A shows human coronary arteries stained with antibodies specific to BMP4 (FIGS. 3A and 3D), von Willebrand factor (FIGS. 3B and 3E), ICAM-1 (FIGS. 3F), and non-immune mouse IgG (NI-IgG) (FIGS. 3C). FIGS. 3A, 3B and 3C are serial sections obtained from minimally diseased (normal) arterial samples, while FIGS. 3D, 3E and 3F are from that showing foam cell lesions (marked as * in FIG. F based on CD68 staining, data not shown). M: medial smooth muscles. Note strong stainings for BMP4 and ICAM-1 in overlapping patches (arrow heads in FIGS. 3D and 3F) in the serial sections. Endothelial cells are marked with arrow heads (FIGS. 3B and 3E).

As shown in (FIG. 3B), one exception was found in the endothelial cells (arrows) overlying foam cell lesions which was stained strongly against the BMP4 antibody. As shown in FIG. 3C, isotype matched non-specific mouse IgG used as a negative control further supported the specificity of BMP4 staining. In contrast, the medial smooth muscle cells and macrophages (FIGS. 3A and D) were most intensely stained against a monoclonal BMP4 antibody (smooth muscle cells and macrophages identified by a-actin and CD-68 staining, respectively, data not shown). To verify the identity of endothelial cells, the serial sections were stained with a von Willebrand factor antibody (endothelial marker, FIGS. 3B and E), demonstrating the location of BMP4 staining in select areas of endothelium. Furthermore, immunostaining with an ICAM-1 antibody showed that the expression of this pro-inflammatory adhesion molecule was selectively increased in the similar endothelial areas expressing BMP4 as marked with arrow heads in FIGS. 3D and F. On the other hand, VCAM-1 was not detected in the adjacent serial sections (data not shown). This result is consistent to the previous finding reported by Endress et al. (7).

### Example 3

### BMP4 produced in endothelial cells by OS stimulates monocyte adhesion.

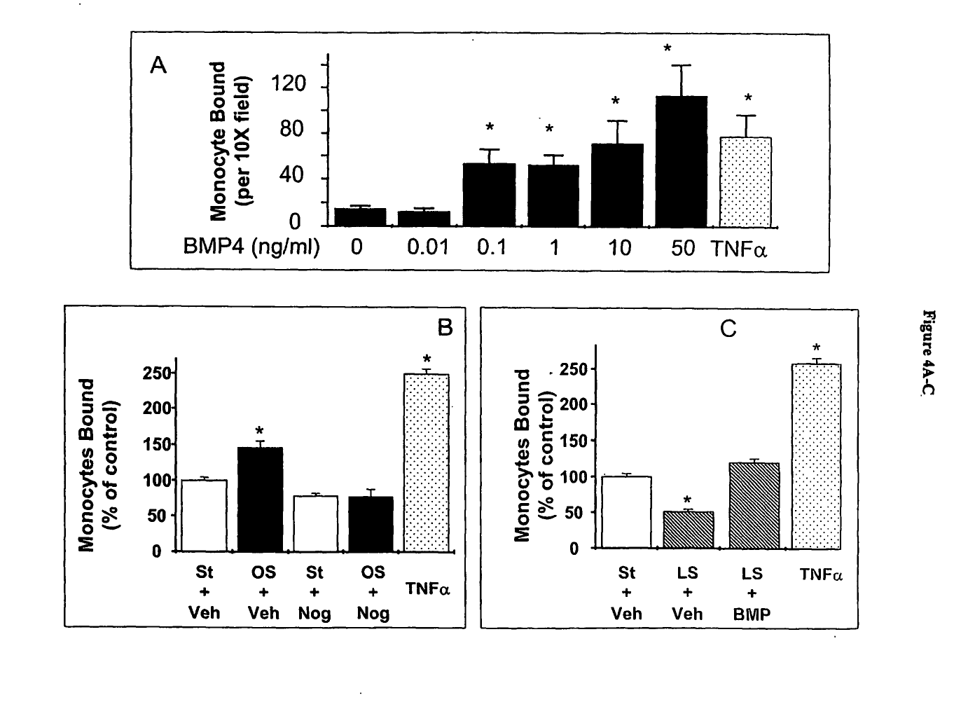

The role of BMP4 in the inflammatory responses observed in lesion-prone areas was investigated. To begin to test the hypothesis, MAEC were treated with increasing amounts of BMP4 for 24 hr, and then monocyte adhesion to endothelium was determined. As a positive control, some cells were treated with a well-known inflammatory cytokine TNF-α (100 U/ml). BMP4 stimulated monocyte binding in a concentration dependent manner with a maximum activation of 4 to 7-fold over control (FIG. 4A, p<0.05). In FIG. 4A to MAEC that were treated with increasing concentrations of BMP4 overnight, BCECF-labeled THP-1 monocytes were added to determine monocyte adhesion. The Bar graph represents mean numbers of bound monocytes per 10× objective field (6-12 different fields per dish) mean±SEM (n=4-6). As a positive control, monocyte binding was determined using MAEC treated with TNF-α (100 U/ml for 2 hrs). As low as 0.1 ng/ml of BMP4 induced a statistically significant increase while 50 ng/ml of BMP4 induced a maximum effect. A similar effect of BMP4 on monocyte adhesion was also observed by transfecting MAEC or human aortic endothelial cells (HAEC) with a vector expressing mouse BMP4 (data not shown).

The role of BMP4 in OS induction of monocyte adhesion in endothelial was determined using a BMP4 inhibitor, noggin (17,18). Exposure of endothelial cells to OS for 24 hr significantly increased monocyte adhesion (FIG. 4B, p<0.05). Treatment of MAEC with noggin (50 ng/ml) inhibited OS-induced monocyte adhesion (FIG. 4B). In FIG. 4B MAEC were exposed to OS or static condition in the presence or absence of noggin or vehicle (veh), and monocyte adhesion determined. Shown bar graph represents the numbers of bound monocytes expressed as % of static control (mean±SEM, n=4) (*p<0.05).

In contrast, exposure of MAEC to LS for 24 his inhibited monocyte adhesion by approximately 50% of static control level (FIG. 4C, p<0.05) as expected (19,20). In FIG. 4C MAEC were exposed to LS in the presence of recombinant BMP4 or vehicle control, followed by monocyte binding assay. Data are expressed as in FIG. 4B (mean±SEM, n=4) (*p<0.05). Since LS exposure significantly inhibited BMP4 expression in endothelial cells (Fig, 2), reversal of the inhibitory effect of LS on monocyte adhesion by BMP4 addition was investigated. For this study, MAEC were exposed in the presence of BMP4 during shear or static control for 24 hrs followed by monocyte adhesion assay. The inhibitory effect of LS on monocyte adhesion was lost when MAEC were sheared in medium supplemented with BMP4 (FIG. 4C, p<0.05). Taken together, these results indicate that BMP4 produced from endothelial cells by OS exposure leads to monocyte adhesion.

### Example 4

### BMP4 Stimulates Monocyte Adhesion by Inducing ICAM-1 Expression in an NFκB-Dependent Manner

Next, the mechanism by which BMP4 increases the monocyte adhesion to endothelial cells was examined. Adhesion of monocytes to endothelial cells is mediated by sequential coordinated molecular interactions between the integrins expressed on monocyte surface and several adhesion molecules expressed on the endothelial surface including ICAM-1, VCAM-1, and E-selectin (Ross, R. (1999) N Engl J Med 340, 115-126; Libby, P., et al. (2002) Circulation 105, 1135-1143). Moreover, it has been shown previously that expression of ICAM-1, VCAM-1, E-selectin on endothelial cell surface is increased in atherosclerosis-prone areas (Ross, R. (1999) N Engl J Med 340, 115-126; Cybulsky, M. I., et al. (2001) J Clin Invest 107, 1255-1262; Endres, M., et al. (1997) Stroke 28, 77-82). ICAM-1, VCAM-1, and E-selectin endothelial expression was examined to determine whether expression was modified in response to OS by FACS analysis.

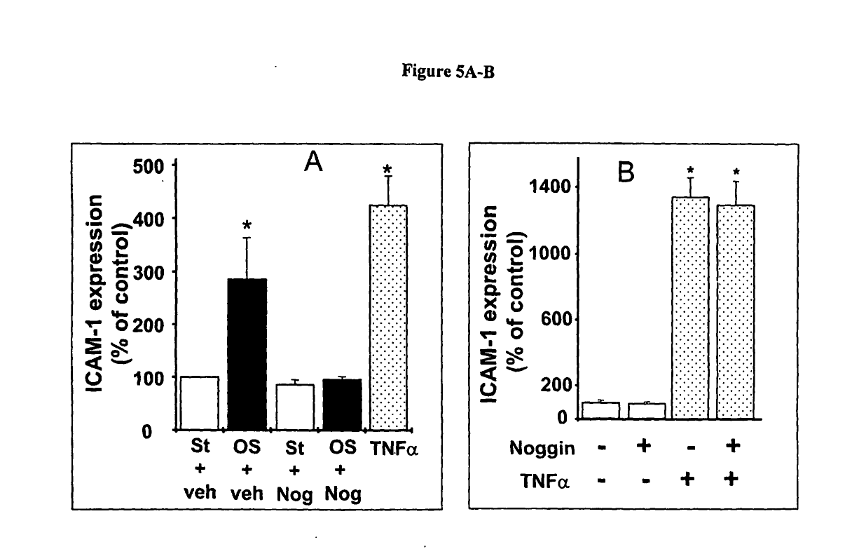

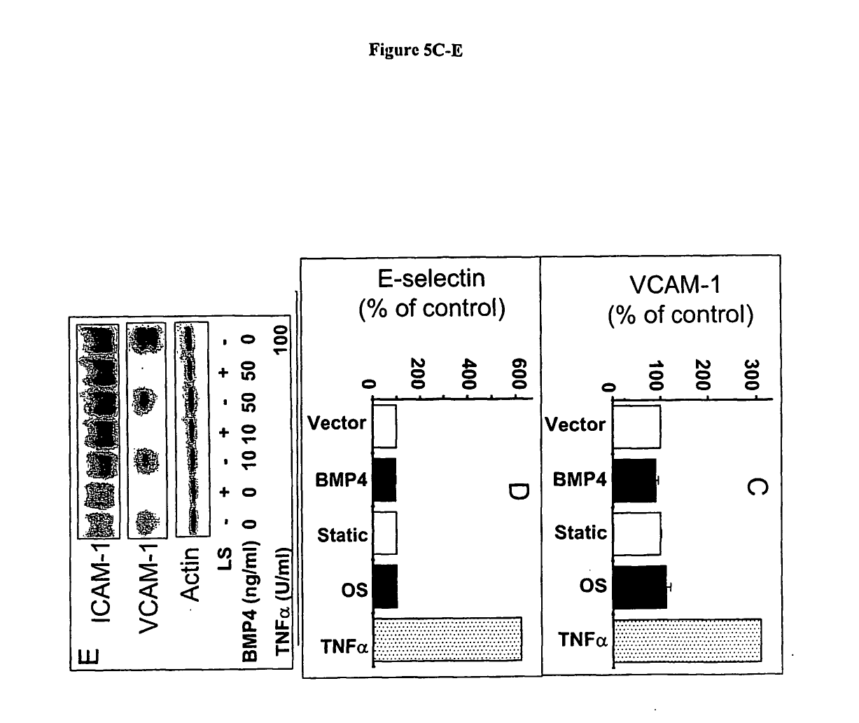

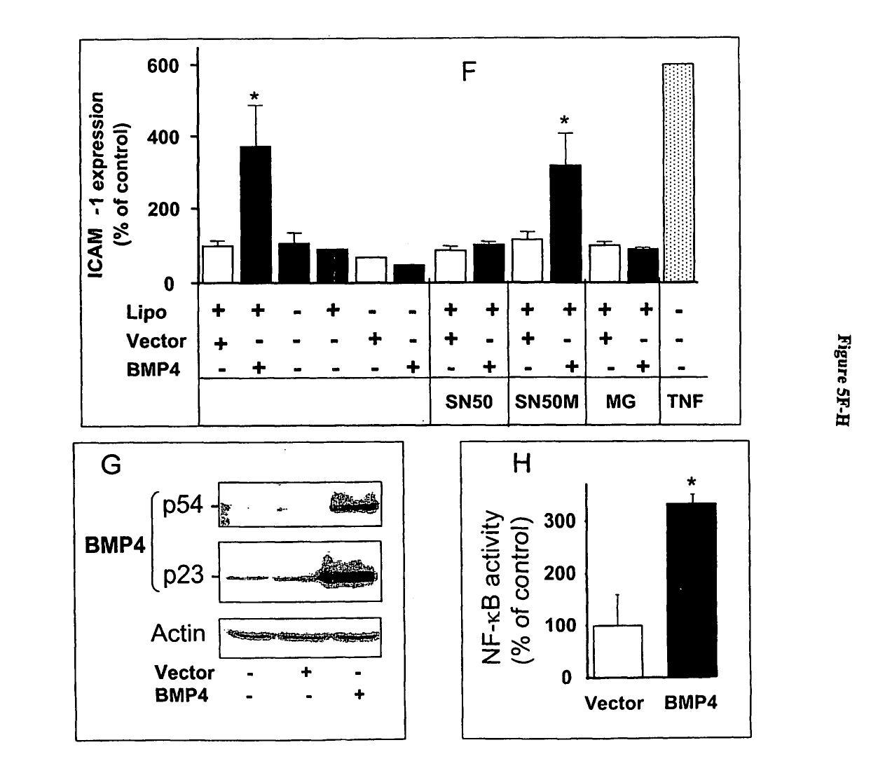

Exposure of HAEC to OS (1 day) increased ICAM-1 expression by 2,8-fold above control (FIG. 5A, p<0.05). For comparison, TNF-α stimulated ICAM-1 expression by 4 to 5-fold above control cells (Fig. SA). To determine whether ICAM-1 expression induced by OS was mediated by a BMP4-dependent mechanism, HAEC were either exposed to OS or static conditions in the presence of a natural BMP antagonist noggin (50 ng/ml). In FIG. 5A after exposing HAEC to OS in the presence or absence of noggin or vehicle control, expression of ICAM-1 was determined by FACS analysis, The mean of log fluorescence values were obtained and expressed as % of static control values, and the bar graph represents mean±SEM, n=4 (*p<0.05). As a positive control, HAEC were treated with TNF=α (100 U/ml, for 2 hr). Noggin completely inhibited ICAM-1 expression in cells exposed to OS, demonstrating that BMP mediates the OS effect (FIG. 5A). In contrast, noggin did not affect ICAM-1 expression if it was induced by a non-BMP family member TNF-α (FIG. 5B. p<0.05) providing further support for the specificity of noggin toward BMP. In FIG. 5B HAEC were treated with TNF-α (100 U/ml for 6 hr) in the presence or absence of noggin (50 ng/ml), followed by FACS analysis to determine ICAM-1 expression as in FIG. 5A (mean±SEM, n=3, *p<0.05).

Unlike ICAM-1, however, neither OS nor BMP4 had any effect on the surface expressions of VCAM-1 and E-selectin in endothelial cells, while TNF-α strongly increased both (FIGS. 5C and D). HAEC treated with OS, BMP4 or static condition were analyzed by FACS using antibodies specific to VCAM-1 (C) and E-selectin (D) as described in panel A using TNFa (1OOU/ml, for 2 hr) as a positive control.

Next, the effect of LS on endothelial expression of ICAM-1, VCAM-1, and E-selectin was examined. Unlike OS, LS significantly decreased VCAM-1 expression without any significant effect on ICAM-1 (FIG. 5E) or E-selectin (data not shown). Unlike ICAM-1, BMP4's effect on VCAM-1 was marginal at best and not significant (p>0.05, n=3). These results suggest that LS can inhibit monocyte adhesion directly by downregulating VCAM-1 expression.

To further investigate the role of BMP4 in ICAM-1 induction, HAEC were transfected with BMP4 vector or an empty control vector by a Lipofectamine-mediated method. BMP4 stimulated ICAM-1 expression by 3 to 4-fold above that of vector control cells (n=3, p <0.001) (FIG. 5F). As further controls, HAEC were mock-transfected with each transfection component, Lipofectamine alone, empty vector alone, BMP4 vector alone, or not transfected at all. ICAM-1 expressions in all mock-transfected cells were similar to untransfected cells (FIG. 5F). These results demonstrate the specific effect of BMP4 on ICAM-1 induction.

Transfection with BMP4 in HAEC increased expression of both BMP4 precursor (p54 found in cell lysate) and the secreted form (p23 found in conditioned medium) in comparison to controls vector as demonstrated by Western blot (FIG. 5G). BMP4 acts as an inflammatory factor by stimulating a specific adhesion molecule, ICAM-1. Whether OS and BMP4 regulate ICAM-1 induction by an NFκB-dependent mechanism was examined (Breuss, J. M., et al. (2002) Circulation 105, 633-638; Cejna, M., et al. (2002) Radiology 223, 702-708; Zahler, S., et al. (2000) Faseb J 14, 555-564). HAEC were exposed to OS, BMP4 or static conditions in the presence or absence of a NF-κB translocation inhibitor SN50 (Lin, Y. Z., et al. (1995) J Biol Chem 270, 14255-14258) or the inactive peptide SN50M or the proteosome inhibitor MG132 (Huang, Y., et al. (2002) J Immunol 169, 2627-2635). Either SN50 or MG132 completely prevented ICAM-1 expression induced by BMP4, whereas SN50M did not inhibit ICAM-1 induction induced by BMP4 (FIG. 5F). These results indicate that NF-κB plays a role in ICAM-1 induction through BMP4.

To demonstrate further whether BMP4 directly stimulates NF-κB activity, the effect of BMP4 on NF-κB activation using a NF-κB reporter construct was investigated. In this study, NF-κB-SEAP construct expressing a secreted form of placental alkaline phosphatase driven by 4 κB sequences in tandem was co-transfected with either BMP4 vector or empty vector control by a Lipofectamine method used above (FIG. 5F). As shown in FIG. 5H, expression of BMP4 stimulated NF-κB activity by more than 3 fold above controls (n=3, *p<0.05).

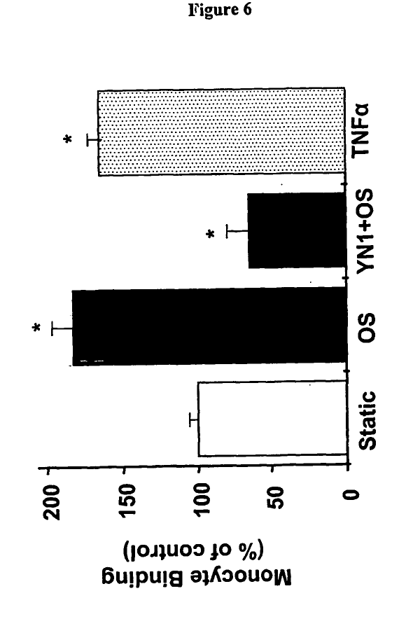

Finally, whether OS-induced monocytc binding was ICAM-1 dependent was studied by using a blocking antibody. As shown in FIG. 6, monocyte adhesion induced by OS was prevented by treating MAEC with an ICAM-1 blocking antibody. In FIG. 6 confluent monolayers of MAEC were exposed to static conditions or OS for 24 hrs with or without 5 μg/ml of the blocking antibody YN1. Following shear stress, monocyte adhesion was determined as described above. TNF-α (100 U/ml for 24 hrs) was used as a positive control Shown data is mean±SEM (n=3, *p<0.01). These results further demonstrate that chronic exposure of endothelial cells to OS induces monocyte adhesion in an ICAM-1 dependent manner.

### Example 5

### Treatment of Endothelial Cells with BMP4 Stimulates Monocyte Adhesion by the ICAM-1 and ROS-Dependent Mechanisms

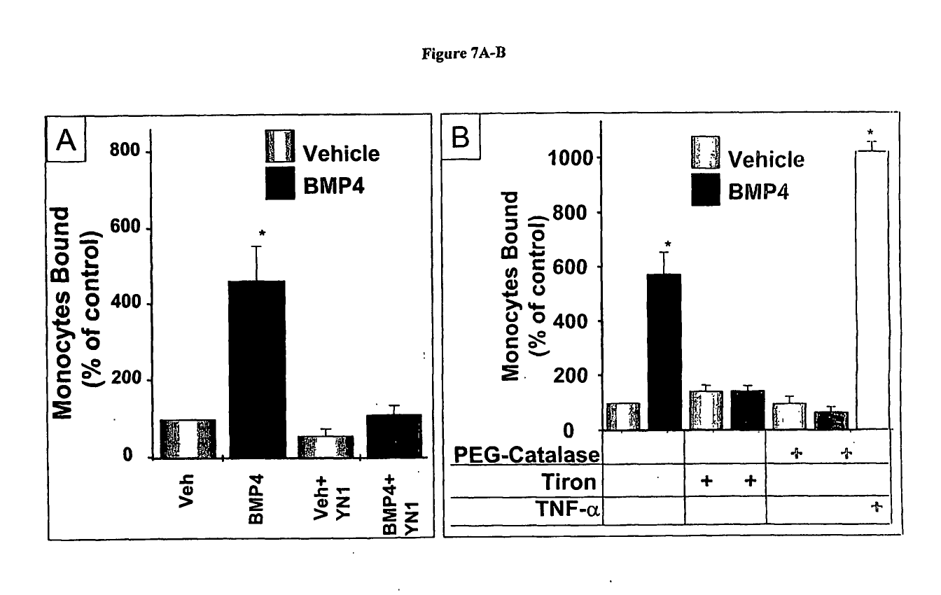

Treatment of MAE-wt cells with BMP4 for 18 hrs stimulated monocyte adhesion by 5 to 6-fold above that of vehicle control, which was completely prevented by YN1 antibody specific for ICAM-1 (FIG. 7A). In FIG. 7A, confluent MAE-wt cells were treated with BMP4 (20 ng/ml) or vehicle for 18 hrs. During the last 30 min of the incubation, 5 μg/ml of the blocking antibody YN1 was included. This data indicates that ICAM-1 expressed on endothelial cell surface plays a critical role in BMP4-induced monocyte adhesion.

Next, the role of reactive oxygen species (ROS) in BMP4-induced monocyte adhesion was determined by treating endothelial cells with ROS removing agents. As shown in FIG. 7B, treatment of MAE-wt cells with the cell-permeable PEG-catalase (converting H₂O₂ to H₂O) and Tiron (scavenging O₂⁻) completely blocked BMP4-induced monocyte binding, demonstrating a role for ROS. TNF-α, a well-known inducer of monocyte adhesion, was used in these studies as a positive control (FIG. 7B).

In FIG. 7B, confluent MAE-wt cells were pre-incubated with cell-permeable PEG-catalase (Catalase, 100 U/ml for 18 hrs) or Tiron (10 μmol/L for 1 hr). Cells were then incubated with 20 ng/ml BMP4 or vehicle for additional 18 hrs. The treated cells in 7A and 7B were washed, and fluorescently-labeled THP-1 monocytes were added. Unbound THP-1 cells were washed after 30 min, and bound monocytes were counted with a fluorescence microscope using a 10× objective (5-10 different fields per dish). The bar graph shows the numbers of bound monocytes expressed as % of control (mean±SEM, n=6 in A and n=4 in B) (*p<0.05). As a positive control, monocyte binding was determined using MAE-wt treated with TNF-α(100 U/ml for 2hrs).

### Example 6

### OS and BMP4 Induce ICAM-1 Expression in a ROS-Dependent Manner

While OS was shown to stimulate monocyte adhesion by inducing ICAM-1 expression on endothelial cell surface in a ROS-dependent manner, it was not known whether BMP4 would also induce ICAM-1 by the same mechanism. To address this question, the role of ROS in OS-induced ICAM-1 expression was determined by using ROS scavengers. Exposure of HAEC to OS for 1 day increased surface expression of ICAM-1 by 68% over static conditions (FIG. 8A). Inclusion of PEG-catalase or Tiron during shear exposure completely prevented the OS-induced ICAM-1 expression, demonstrating a role of ROS in this response (FIG. 8A).

The role of ROS in BMP4-dependent ICAM-1 expression on endothelial cell surface was investigated. Transfection of BMP4 in HAEC produces both pre-propeptide (54 kDa) and an active, secreted form (23 kDa) of BMP4 as determined by Western blot analysis using a monoclonal BMP4 antibody. BMP4 overexpression stimulated ICAM-1 expression by 84% above that of empty vector control (FIG. 8B). BMP4-induced ICAM-1 expression was inhibited by either PEG-catalase (26% of control) or N-Acetyl-cysteine (23% of control), suggesting an important role for ROS.

### Example 7

### NAD(P)H Oxidases Plays a Role in BMP4-Induced Inflammatory Response of Endothelial Cells

To identify a specific molecular source of ROS production MAE cells were cultured from p47phox null mice (MAE-p47^{-/-}). Using MAE-p47-1-cells and a ESR spin trap CMH, OS was shown to stimulate monocyte adhesion by a mechanism dependent on ROS derived from p47phox-based NAD(P)H oxidases. Moreover, the inflammatory responses of MAE-^{p474-/-} cells were “rescued” by transfecting them with p47phox cDNA.

The same approach was used to determine whether ROS-derived from NAD(P)H oxidases play a role in monocyte adhesion to endothelial cells. As shown in FIG. 9A, MAE-p47^{-/-} cells expressed no detectable amount of p47phox. However, transfection of the cells with p47phox cDNA led to overexpression of the protein. Actin blot was used as an internal control in this study, showing an equal protein loading (FIG. 9A).

More particularly, FIG. 9 shows MAE-p47^{-/-} cells transfected with p47phox or an empty vector (Vector) control by using Lipofectamine 2000 (Lipo) In FIG. 9A one day after transfection, cell lysates were obtained and analyzed by Western blot using a p47 specific antibody. The same blot was stripped and re-probed with an actin antibody as a protein loading control in each lane. The shown blot is a representative of 4 independent experiments.

Next, monocyte adhesion was determined in these cells. As shown previously, treatment of MAE-p47^{-/-} cells with BMP4 failed to stimulate monocyte adhesion (compare lanes 1 and 2, FIG. 9B). Transfected cells were treated with BMP4 (20 ng/ml), TNF-α (100 U/ml) or vehicle control. For control purposes, some cells were transfected with lipofectamine alone or vectors alone. After 18 hrs, cells were washed and monocyte binding was determined as described in FIG. 7. The bar graph shows the mean±SEM, n-4, (*p<0.05). Overexpression of p47phox protein in the MAE-p47^{-/-} cells “rescued” the monocyte adhesion response, showing 4- to 5-fold increase above the control (compare lanes 7 and 8, FIG. 9). To ensure the rescued response was specifically due to p47phox transfection, several negative controls were performed by treating cells with Lipofectamine alone (lane 3), TNF-α alone (lane 4), and Lipofectamine+ empty vector with or without BMP4 (lanes 5 and 6). In addition, as a positive control, treatment of the “rescued” cells with TNF-α showed approximately 4-fold increase in monocyte adhesion (lane 9). These results demonstrate that BMP4 induces monocyte adhesion in a NAD(P)H oxidase-dependent manner.

### Example 8

### BMP4 Stimulates O₂⁻ and H₂O₂ Production in Endothelial Cells

Since OS is well known to stimulate production of ROS including O₂⁻ and H₂O₂, whether BMP4 alone could also stimulate the ROS production in MAE cells was examined. As shown in FIG. 10A, treatment of MAE-wt cells with BMP4 for 18 hrs stimulated 0₂⁻ production by 2.9-fold above control as determined by ESR spectroscopy of CM. This assay was performed in the presence or absence of SOD to quantify the SOD-inhibitable amount of CM, providing further specificity for the assay.

In FIG. 10A immediately following treatment of MAE wt cells with BMP4 (200 ng/ml) for 18 hrs, cells were trypsinized and reconstituted in Krebs-Hepes buffer. Equal number of cells was incubated with a spin trap CMH in the presence or absence of PEG-SOD (50 U/mL). The specific O₂⁻ production was calculated by subtracting SOD-inhibitable portions from total O₂⁻ production as determined by following CM production (mean±SEM, n=3, *p<0.05).

In addition, BMP4 stimulated production of H₂O₂ by 70% above control as determined by oxidation of DCF-DA (FIG. 10B). BMP-dependent H₂O₂ production was completely blocked if MAE-wt were incubated with cell-permeable PEG-catalase (100 U/ml for 1 day) before and during BMP4 treatment, demonstrating the specificity of the assay (FIG. 10B). The cells were washed, incubated with DCF-DA, and the fluorescent intensities determined by fluorometry. Shown is a mean±SEM (n=3, p<0.05). In this assay, a redox cycling menadione (10 μM) was used as positive control (data not shown). These results demonstrate that BMP4 alone, much like OS, can stimulate production of O₂⁻ and in endothelial cells.

### Example 9

### OS-Induced O₂⁻ Production is Mediated by BMP4

So far, we have shown that either OS or BMP4 can stimulate ROS production in endothelial cells. These parallel findings raise an important question whether BMP4 is responsible for the ROS production triggered by OS in endothelial cells. To answer this question, it was determined whether blocking BMP4 with its antagonist noggin can prevent O₂⁻ production induced by OS in MAE-wt. As shown in FIG. 11, exposure of MAE-wt to OS for 1 day increased O₂⁻ level by 2.8-fold above static controls. Treatment of the cells with noggin during OS exposure virtually blocked the O₂⁻ increase (FIG. 11). This result provides strong evidence that BMP4 induced by exposing endothelial cells to OS plays a critical role in ROS production.

### Example 10

### BMP Signal Transduction Pathway

FIG. 12 is a schematic showing endothelial cells in lesion-prone areas (marked with a thick line) are exposed to disturbed flow conditions including OS. Exposure of endothelial cells to OS induces BMP4 expression which stimulates production of ROS in a manner dependent on p47phox-dependent NAD(P)H oxidases. ROS is then produced, which in turn is proposed to activate NFκB. Activated NFκB stimulates ICAM-1 expression and monocyte binding and recruitment, leading to foam cell formation and atherosclerosis. In contrast, LS acts as a potent anti-inflammatory and anti-atherogenic force by mechanisms including down-regulation of BMP4 and VCAM-I.

If we compare the OS effects on expression of BMP4 and ICAM-1 as well as monocyte adhesion to those of LS instead of static conditions, the huge effects of the pro-atherogenic force become more pronounced clearer than what is shown by the data. For example, endothelial cells exposed to LS express BMP4 mRNA and protein at almost undetectable levels. Exposure to OS, however, dramatically upregulated expression of BMP4 mRNA and protein (FIG. 2). This is consistent to human coronary artery data showing that BMP4 expression is undetectable in normal (minimally diseased) arteries, while it is strongly expressed in endothelial patches overlying foam-cell lesions. In typical studies, OS increases monocyte binding 8 to 10-fold above that of LS (data not shown).

The present disclosure presents most of the OS and LS results in comparison to static control conditions. However, it may be more appropriate to compare OS effects to LS conditions rather than to static conditions. Currently, most investigators in this field use static cultured cells as a physiologically “normal” control. This is necessary due to technical constraints of culturing cells under continuous shear conditions. The majority of arterial endothelial cells in vivo, however, are continuously exposed to LS and aligned to the direction of the flow. In contrast, endothelial cells in the hemodynamically defined lesion-prone areas are exposed to unstable and low wall shear conditions. Therefore, endothelial cells cultured under “static” conditions may represent low or no-shear conditions and may not represent a true “control” conditions. Many in vivo studies examine the differences between the normal regions exposed to LS in straight arteries and the lesion-prone areas exposed to disturbed and low shear conditions in branched or curved arteries.

All patents, patent applications, books and literature cited in the specification are hereby incorporated by reference in their entirety. In the case of any inconsistencies, the present disclosure, including any definitions therein will prevail. The subject matter has been described with reference to various specific and preferred embodiments and techniques. However, it should be understood that many variations and modifications may be made while remaining within the spirit and scope of the invention. It is therefore to be understood that, within the scope of the appended claims, the invention may be practiced otherwise than as specifically described herein.

What is claimed is:

1. A pharmaceutical composition comprising a bone morphogenic protein antagonist or a prodrug thereof in an amount sufficient for inhibiting vascular inflammation by interfering with binding of bone morphogenic protein or a fragment thereof to bone morphogenic protein receptors.

2. A pharmaceutical composition comprising a bone morphogenic protein receptor antagonist or a prodrug thereof in an amount sufficient to inhibit or reduce vascular inflammation by interfering with bone morphogenic protein binding to bone morphogenic protein receptors.

3. A pharmaceutical composition comprising a modified bone morphogenic polypeptide or a prodrug thereof in an amount sufficient for inhibiting vascular inflammation by competitively inhibiting binding of bone morphogenic protein to endothelial bone morphogenic protein receptors, wherein binding of said modified bone morphogenic protein to said bone morphogenic protein receptor does not activate said receptor.

4. The composition of claims 1-3, wherein the bone morphogenic protein receptors are vascular cell bone morphogenic protein receptors.

5. The composition of claim 4, wherein the bone morphogenic protein antagonist comprises a polypeptide of noggin, chordin, DAN, or veinless.

6. The composition of claims 1-3, wherein the bone morphogenic protein antagonist consists of the N-terminal fragment of noggin, chordin, DAN or veinless.

7. The compositions of claims 1-4, wherein the bone morphogenic protein is bone morphogenic protein 4.

8. The compositions of claims 1-5, further comprising a second therapeutic agent.

9. The compositions of claim 8, wherein the second therapeutic agent comprises an anti-inflammatory agent, cholesterol lowering agent, or a combination thereof.

10. A vector comprising a promoter operably linked to polynucleotide encoding a modified bone morphogenic polypeptide that binds to a bone morphogenic protein receptor without activating said receptor.

11. The vector of claim 10, wherein the promoter is an inducible promoter.

12. The vector of claim 11, wherein the promoter is induced in vascular cells.

13. The vector of claim 12, wherein the vascular cells are endothelial cells.

14. The composition of claims 1-13 further comprising a pharmaceutically acceptable carrier.

15. A medical device comprising a bone morphogenic protein antagonist, a bone morphogenic protein receptor antagonist, or a combination thereof.

16. The medical device of claim 15, wherein the device is a vascular stent.

17. The device of claims 15-16, wherein the device releases an amount of antagonist sufficient to inhibit or reduce vascular inflammation by interfering with or reducing the binding of bone morphogenic protein or a fragment thereof to a bone morphogenic protein receptor.

18. The device of claims 15-17, wherein the device is configured to be inserted into a blood vessel.

19. The device of claim 15-18, wherein the release of antagonist is sustained over a period of time.

20. A method of decreasing or inhibiting monocyte adhesion to vascular cells comprising inhibiting binding of bone morphogenic polypeptide to said vascular cells by contacting bone morphogenic polypeptide present in vascular fluid or tissue in contact with said vascular cells with a bone morphogenic polypeptide antagonist in an amount sufficient to inhibit or reduce the expression of cell adhesion molecules by said vascular cells.

21. A method of inhibiting a vascular inflammatory response comprising contacting extracellular vascular fluid with an amount of bone morphogenic protein antagonist sufficient to inhibit binding-of bone morphogenic protein to vascular cells in contact with said vascular fluid.

22. A method of inhibiting vascular inflammation comprising contacting vascular cells with a bone morphogenic protein receptor antagonist in an amount sufficient to inhibit or reduce binding of bone morphogenic protein to said vascular cells.

23. A method of inhibiting vascular inflammation comprising contacting vascular cells with an inhibitory polynucleotide specific for a bone morphogenic polypeptide or bone morphogenic protein receptor.

24. A method for treating vascular inflammation comprising administering to a host an amount of bone morphogenic protein antagonist or bone morphogenic protein receptor antagonist sufficient to inhibit binding of bone morphogenic protein or a fragment thereof to vascular cells of the host.

25. A method for treating atherosclerosis comprising administering to a host an amount of bone morphogenic protein antagonist or bone morphogenic protein receptor antagonist sufficient to inhibit binding of bone morphogenic protein or a fragment thereof to vascular cells of the host.

26. A method for treating vascular inflammation comprising administering to a host an amount of bone morphogenic protein antagonist or bone morphogenic protein receptor antagonist sufficient to inhibit binding of bone morphogenic protein or a fragment thereof to vascular cells of the host and inhibit or reduce the expression of cell surface adhesion polypeptides.

27. The method of claims 20-26, wherein binding of bone morphogenic protein to bone morphogenic receptors is reduced or inhibited.

28. The method of claims 20-27, wherein the bone morphogenic protein antagonist comprises a polypeptide of noggin, chordin, DAN, or veinless.

29. The method of claim 28, wherein the bone morphogenic protein antagonist consists of an N-terminal polypeptide fragment of noggin, chordin, DAN or veinless.

30. The methods of claims 20-29, wherein the bone morphogenic protein is bone morphogenic protein 4.

31. The methods of claims 20-30, further comprising a second therapeutic agent.

32. The method of claim 32, wherein the second therapeutic agent comprises an additional anti-inflammatory agent, cholesterol lowering agent, or a combination thereof.

33. A method for treating vascular inflammation comprising inserting the medical device of claims 15-19 into a vascular conduit of a host.

34. A pharmaceutical composition comprising a bone morphogenic protein antagonist or a prodrug thereof in an amount sufficient for inhibiting vascular inflammation by interfering with binding of bone morphogenic protein or a fragment thereof to bone morphogenic protein receptors, wherein the antagonist includes at least a portion of a polypeptide corresponding to any one of SEQ. ID. Nos. 1-10.
